(19) United States
(12) Patent Application Publication
(10) Pub. No.: US 2017/0217976 A1
(43) Pub. Date: Aug. 3, 2017

(54) 6-(AMINO ACID)-MORPHINAN DERIVATIVES IN COMBINATION WITH PERMEATION ENHANCERS FOR USE AS AN ORALLY, RECTALLY, TRANSDERMALLY OR NASALLY ADMINISTERED MEDICAMENT

(71) Applicant: CHIRONWELLS GMBH, Innsbruck (AT)

(72) Inventor: Helmut SCHMIDHAMMER, Innsbruck (AT)

(21) Appl. No.: 15/127,681

(22) PCT Filed: Mar. 23, 2015
(86) PCT No.: PCT/EP2015/000624

§ 371 Date: Sep. 20, 2016

(30) Foreign Application Priority Data
Mar. 21, 2014 (EP) 14001054

(51) Int. Cl. C07D 489/02

(52) U.S. Cl. CPC C07D 489/02

(57) ABSTRACT

The present invention relates to a composition for use in an orally, or rectally, transdermally or nasally administered medicament, characterized in that it includes: (b) at least one compound of Formula (I) and (b) at least one permeation enhancer, selected from the group consisting of saturated and/or unsaturated organic fatty acids, or pharmaceutically and pharmacologically acceptable salts thereof, and thiomers. It further relates to a pharmaceutical formulation comprising said composition. It furthermore relates to bioreversible esters of compounds of Formula (I).

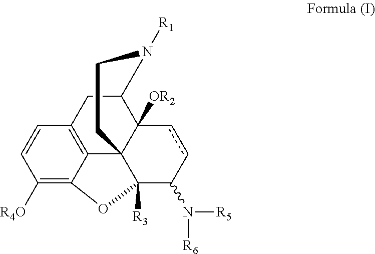

The present invention relates to a composition for use as an orally, rectally, transdermally and/or nasally administered medicament comprising at least one 6-(amino acid)-morphinan compound and at least one permeation enhancer. The invention also relates to a pharmaceutical formulation comprising said composition (Embodiment 1). The invention further relates to bioreversible esters of 6-(amino acid)-morphinan compounds for use in an orally, rectally, transdermally and/or nasally administered medicament (Embodiment 2).

Opioid analgesics play a central role in pain control and are generally considered to be highly effective in the management of moderate-to-severe pain.

They can be classified into three classes: natural derivatives occurring in opium such as morphine and codeine; partially synthetic derivatives, including hydromorphone, oxycodone, oxymorphone, and buprenorphine; and synthetic compounds such as levorphanol, butorphanol, fentanyl, sufentanil, and tapentadol.

Opioids act on three G-protein-coupled receptors (GPCRs) that is, μ (MOR), δ (DOR), and κ (KOR), but it appears that the analgesic action of the most commonly used opioid analgesics is mediated primarily via the MOR. Activation of MORs, widely expressed in the central nervous system (CNS) and peripheral tissues, is responsible not only for beneficial (analgesia) effects but also for a number of several centrally mediated adverse effects, which limits their clinical usefulness [H. Schmidhammer et al., International Journal of Medicinal Chemistry 2012, Article ID 208039, 10 pages, doi:10.1155/2012/208039]. Adverse effects associated with opioid analgesics include respiratory depression, nausea, sedation, dizziness, vomiting, hypotension, and constipation. Long-term opioid use can cause tolerance, and thus complicating optimal pain treatment. Another concern with prolonged use of opioids is physical dependence and development of addictive disorders.

As it is the case for other GPCRs, also MOR, DOR and KOR agents with agonistic effect can be distinguished from those with antagonistic effect. An agonist is an agent that binds to a receptor and activates that receptor in order to mimic the action of the naturally occurring, endogenous transmitter-molecule. A therapeutically used agonist typically has the same or a stronger affinity to the respective receptor than the endogenous transmitter-molecule. An antagonist, on the other hand, is an agent that binds to a receptor but does not elicit the response that the endogenous transmitter-molecule would trigger. Instead, the antagonist blocks the receptor and prevents its activation by endogenous transmitter-molecules or agonistic drugs.

Opioids, morphine and codeine, for example, act as classical agonists. When morphine enters the brain, it binds to opioid receptors and activates them. In case of a morphine overdose, where the high dose of morphine may cause respiratory depression and a drastic drop in blood pressure and heart rate, one may administer naloxone, an opioid antagonist. Naloxone competes with morphine for binding to the receptors, but with a higher affinity than morphine and thus, replaces much of the morphine at the respective receptors. In cases of opioid-addiction, naloxone can be used for rapid detoxification under anaesthesia which masks the acute withdrawal syndrome. A third class of opioids comprises drugs acting as a partial agonist at a single receptor (e.g.: buprenorphine) or as agonist or partial agonist at one receptor and as an antagonist at another (e.g.: the weak MOR-antagonists and partial KOR agonists: pentazocine, butorphanol, nalbuphine). These drugs can be classified as nalorphine-like or morphine-like. And finally there are those, which do not comply with either of the classifications and form a separate class (e.g.: meptazinol).

There are three traditional types of pharmacotherapy for opioid addiction: (i) Antagonists that bind to the opioid receptors with higher affinity than agonists but do not activate the receptors (e.g.: naloxone, naltrexone, nalmefene), (ii) agonists (e.g.: methadone), (iii) partial agonists (e.g.: buprenorphine), (iv) and other agents (e.g.: clonidine) to help withdrawal from opioid drugs as a means of entry into treatment. Some opioid antagonists are not pure antagonists but in fact do produce some weak opioid partial agonist effects, and can produce analgesic effects when administered in high doses to opioid-naive individuals. Examples of such compounds include nalorphine and levallorphan. Some opioids can also have disadvantages such as worsening respiratory depression in patients who have overdosed on non-opioid sedatives such as alcohol, benzodiazepines or barbiturates.

Chemical approaches towards the identification of novel MOR analgesics with reduced CNS side effects are represented by structural modifications of morphinan-6-ones in key positions that are important for binding, selectivity, potencies and efficacies at opiod receptors. A representative example is the development of 14-O-methyl-substituted derivative of the clinically used MOR analgesic oxymorphone, namely 14-O-methyloxymorphone. It was reported that substitution of the hydroxyl group with a methoxy group in position 14 not only increases affinity to opiod receptors while retaining the MOR selectivity of oxymorphone, but also markedly enhances the antinociceptive potency [H. Schmidhammer, L. Aeppli, L. Atwell et al., Journal of Medicinal Chemistry 1984, 27, 1575-1579]. However, this MOR agonist induces the classical opiod unwanted effects of the conventional MOR analgesics [H. Schmidhammer et al., International Journal of Medicinal Chemistry 2012, Article ID 208039, 10 pages, doi:10.1155/2012/208039].

This fact has stimulated the development of structurally modified opioid drugs acting selectively in the periphery and as a result lacking unwanted CNS side effects [C. Stein at al., Nat. Med. 2003, 9, 1003-1008]. Strategies to reduce their access to the CNS include chemical modifications at opiod compounds that increase their hydrophilicity, such as incorporation of highly polar hydrophilic substituents [I. Bileviciute-Ljungar et al., The Journal of Pharmacology and Experimental Therapeutics 2006, 317, 220-227].

Opioid antagonists have therapeutic potential in the treatment of a variety of disorders. These include, for instance, constipation, drug addiction and other behavioural addictions (e.g.: food, buying, internet, computer, phone and gambling addiction), food intake, shock, alcoholism, mental/psychiatric and stress related disorders. The universal opioid antagonist, naloxone, which is a competitive antagonist of all three types of opioid receptors (MOR, KOR and DOR), is used in order to reverse the potentially lethal respiratory depression caused by neurolept analgesia or opioid overdose. Among other pharmacological effects, it antagonizes the blood pressure drop in various forms of shock, reverses neonatal hypoxic apnea, counteracts chronic idiopathic constipation, reduces the food intake in humans and shows beneficial effects in central nervous system injuries. Its analogue naltrexone has considerable and longer duration of action higher oral efficacy, which make it suitable for the management of opioid and alcohol and dependence. The opioid antagonist nalmefene (Selincro®), an analogue of naltrexone, was launched in Europe by Lundbeck in 2013 for the reduction of alcohol consumption in alcohol dependent patients: ClinicalTrials.gov Identifier: NCT00811720.

Peripherally acting opioid antagonists, show for instance therapeutic potential for gut diseases associated with opioid mechanisms or treatments such as post-operative ileus (POI) and opioid-induced bowel dysfunction (OBD) [P. Holzer, Regul. Pept. 2009, 155, 11-17; and other gastrointestinal motility disorders [M. Camilleri, Neurogastroenterol. Motil. 2005, 17, 157-165].

There is emerging evidence that opioid receptor antagonists that are interacting preferentially with peripheral opioid receptors may also have a prokinetic action, reversing pathological states of gastrointestinal hypomotility that are due to overactivitiy of the enteric opioid system. Such a prokinetic action can be envisaged from the experimental observations that naloxone as well as selective MOR opioid antagonists i.e. cyprodime, can per se facilitate propulsive peristalsis [A. Shahbazian, A. Heinemann, H. Schmidhammer et al., Br. J. Pharmacol. 2002, 135, 741-750].

The oral prolonged-release oxycodone/naloxone combination tablet (Targinact®) reduces OBD in patients with chronic severe pain compared to treatment with the opioid analgesic oxycodone alone [P. Holzer, Regul. Pept. 2009, 155, 11-17;]. Opioid analgesia is not impaired by a prolonged-release formulation of oral naloxone since its bioavailability after oral administration is low due to a high first-pass effect in the liver. Nevertheless, it needs to be achieved that naloxone can easily cross the blood-brain barrier and hence reverse analgesia if given in sufficient oral doses that exceed the hepatic capacity [P. Holzer, Regul. Pept. 2009, 155, 11-17]. Thus, the therapeutic range of immediate-release naloxone is rather narrow.

A promising approach to normalize pathologic inhibition of gut function is represented by peripherally acting opioid antagonists. The design strategies of such peripherally active opioid antagonists have focused on developing hydrophilic compounds, thereby diminishing permeability through the lipophilic blood-brain barrier and preventing central antagonism. Methylnaltrexone (Relistor®) and alvimopan (Entereg®) represent two opioid antagonists with activity at peripheral MORs, which show effectiveness in both preclinical and clinical studies in reversing opioid-induced slowing of the gastrointestinal tract without interfering with analgesia [P. Holzer, Regul. Pept 2009, 155, 11-17].

In 2008, the US Food and Drug Administration (FDA) and the European Medicines Agency have approved Relistor® (methylnaltrexone) for subcutaneous injection in the treatment of opioid-induced constipation in patients with advanced illness who are receiving palliative care if response to laxative therapy has not been sufficient. The adverse reactions reported for methylnaltrexone include for example transient orthostatic hypotension and gut-related reactions such as abdominal cramps, flatulence and nausea. Oral dosing of alvimopan (Entereg®) has been approved by the FDA in 2008 for the short-term treatment of POI following bowel resection in hospitalized adult patients [P. Holzer, Regul. Pept 2009, 155, 11-17]. Common side effects include nausea, vomiting, and abdominal discomfort. A one-year phase III trial of patients on opiate therapy for chronic non-cancer pain revealed a numerical imbalance in the number of ischemic cardiovascular events, neoplasms and fractures in patients on alvimopan relative to placebo [P. Holzer, Regul. Pept. 2009, 155, 11-17]. The use of alvimopan is also limited by its low oral bioavailability (approx. 6%) and very low water solubility.

These facts have encouraged the development of peripherally acting 6-(amino acid)-morphinan derivatives as highly potent opioid receptor antagonists, which do not counteract opioid induced analgesia due to the fact that they have very limited access to the CNS, show an improved side effect profile and are well soluble in water.

EP-A1-1762569 relates to a class of 6-(amino)-morphinan derivatives which have been found to show high pharmaceutical efficiency as opioid agonists having analgesic potency and as opioid receptor antagonists. Moreover, their access to the CNS is restricted so that they act preferably peripherally which reduces CNS-derived side effects.

On the other hand, compounds disclosed in EP-A1-1762569 may exhibit an extremely high hydrophilicity. This applies particularly to compounds bearing an amino acid substituent in 6-position of the morphinan skeleton (linked via the N-terminus to the morphinan skeleton) wherein the amino acid moiety is not further substituted i.e. bears a free carboxylic acid group. This class of compounds may be designated as 6-(amino acid)-morphinans, which, due to the free carboxylic acid group, exist in their zwitterionic form, which significantly increases the hydrophilicity and water solubility of these compounds.

Disadvantageously, a high hydrophilicity usually also reduces the oral, rectal, transdermal and nasal bioavailability of pharmaceutically active compounds due to incomplete absorption. Drugs of very low lipid solubility, including those that are strong acids or bases, are generally poorly absorbed from the gut [H. P. Rang H, M. M. Dale, J. M. Ritter, R. J. Flower. In: Pharmacology, 6^{th} Edition. Churchill Livingstone, 2007, 108]. The low bioavailability upon oral, rectal, transdermal and nasal administration complicates and/or decreases their absorption into the bloodstream, thereby diminishing their therapeutic efficiency. On the other hand, oral, rectal, transdermal and/or nasal administration of a therapeutically highly efficient drug is a particularly attractive way to administer a pharmaceutically active agent since it is easy and straight-forward, not time consuming and non-invasive. Moreover, a high bioavailability upon oral, rectal, transdermal and/or nasal administration reduces the frequency of administration and/or allows a reduced dosage of the drug which, at the end, contributes significantly to patient compliance and patient satisfaction.

In view of the aforesaid, it would seem desirable to improve the bioavailability of highly hydrophilic 6-(amino acid)-morphinans upon oral, rectal, transdermal and/or nasal administration in order to benefit from their therapeutic potential as highly active analgesics.

A number of concepts are known in the art to improve the bioavailability of poorly resorbing drugs.

US-A1-2010/0028421, for example, relates to a solid oral dosage form comprising hydrophilic macromolecules such as peptides, proteins, oligosaccharides or polysaccharides as pharmaceutically active ingredients. The document overcomes the low bioavailability of the active agents by including a permeation enhancer in the solid oral dosage form which may be a medium chain fatty acid salt, ester, ether or a derivative of a medium chain fatty acid. However, the document does not disclose any active agents that are comparable in terms of structure and therapeutic effect to morphine-like compounds, such as the 6-(amino acid)-morphinan derivatives of EP-A1-1762569.

U.S. Pat. No. 6,495,120B2 discloses a formulation for the oral administration of large and small molecular weight compounds, peptides, polypeptides, proteins and analgesics such as morphine and fentanyl. An efficient and convenient drug delivery is achieved by including an absorption enhancer in the formulation which may be selected from hydroxypropyl-beta-cyclodextrin and surfactants such as benzalkonium chloride, benzethonium chloride, polysorbate 80, sodium lauryl sulfate, Brij surfactants, Tween surfactants, and Pluronic surfactants. On the other hand, the document does not disclose or suggest a strategy on how to improve the oral and rectal bioavailability of compounds such as 6-(amino acid)-morphinan derivatives of EP-A1-1762569.

According to WO-96/33678, the oral administration of drugs is unsatisfactory for a number of reasons consisting inter alia in poor bioavailability of the drug. The document therefore identifies a need for the provision of an effective and consistent drug delivery system that overcomes the disadvantages of oral administration. For the solution of this problem, it suggests a composition which is sought for the transdermal administration of a basic drug having preferably a pkₐ of about 8.0 or more, such as oxybutynin, scopolamine, fluxetine, epinephedrine, morphine and hydromorphine, through the skin or mucosa. The composition further comprises a permeation enhancer that contributes to the increase of the delivery of the bioactive agent across biological membranes, particularly skin and mucosa, which essentially consists of triacetin (glyceryl triacetate). WO-96/33678 thus does not disclose or suggest a strategy for increasing the oral or rectal bioavailability of a drug but rather its transdermal bioavailability. In addition, the drugs identified in this prior art are basic compounds with low hydrophilicity.

WO-A1-2005/067897 discloses a formulation used in a transdermal delivery device to deliver dihidropyridine-type calcium antagonists through the skin. The formulation comprises as a permeation enhancer a fatty acid such as oleic and linoleic acid which facilitates the absorption of an active agent through the skin. The document does not teach a strategy on how to increase the oral or rectal bioavailability of a dihydropyridine-type calcium antagonist let alone of morphine-like compounds.

According to WO-88/09676, many opioids are known to have a poor bioavailability in the mammalian systemic circulation and the bioavailability of orally administered opioids may be unpredictable since various factors such as changes in acidity and food content may cause changes in the amount of drug absorbed from the gastrointestinal tract. The document suggests a pharmaceutical composition of opioids such as fentanyl, morphine, oxymorphone comprising a permeation enhancer consisting in a saturated or unsaturated fatty acid of 8 to 18 carbons such as linoleic or oleic acid, or a C1 to C4 alkylester thereof as a permeation enhancer.

WO 2008/144888A1 discloses formulations for the oral administration of therapeutic agents. It aims at improving the bioavailability of difficultly soluble agents (i.e. compounds with low water solubility/low hydrophilicity).

Bilericinte-Ljungar et al., J. Pharm. Exp. Ther., Vol. 317, No. 1, 220-227 disclose information on analgesic properties of a μ-Opioid Receptor agonist, sharing an oral bioavailability (relative to the effect achieved by subcutaneous application) of only 1%, i.e. far below any acceptable level.

Summing up the above, there is no concept provided in the art that teaches how to improve the oral, rectal, transdermal and/or nasal bioavailability of 6-(amino acid)-morphinan derivatives. It is therefore the object of the present invention to provide highly hydrophilic 6-(amino acid)-morphinan derivatives in a form wherein the oral, rectal, transdermal and nasal bioavailability of said highly hydrophilic 6-(amino acid)-morphinan derivatives is increased for use as an orally, rectally, transdermally and/or nasally administered medicament.

### Embodiment 1

Said object is solved by a composition for use in an orally, rectally, transdermally or nasally administered medicament, the composition comprising:

(a) at least one compound of Formula (I)

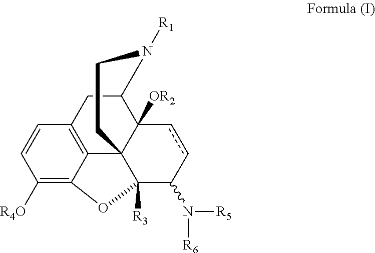

wherein the substituents R₁, R₂, R₃, R₄, R₅ and R₆ are as defined in claim 1;

and/or a pharmaceutically acceptable acid addition or base addition salt thereof, characterized in that the composition further comprises

(b) at least one permeation enhancer, selected from the group consisting of saturated and/or unsaturated organic fatty acids, or pharmaceutically and pharmacologically acceptable salts or esters thereof; thiomers; and further organic compounds, selected from acetone; alcohols, glycols and glycerides such as ethanol, caprylic alcohol, propylene glycol; essential oils such as niaouli oil, eucalyptus oil, Alpinia oxyphylla oil, turpentine oil, sweet basil oil, tulsi oil, cardamom oil, peppermint oil, fennel oil, black cumin oil; terpenes such as geraniol, nerol, linalool, limonene, α-terpineol, β-terpineol, γ-terpineol, menthol, carveol, menthone, pulegone, iso-pulegone, piperitone, carvomenthone, carvone, 1,8-cineole, α-thujene, car-3-ene, α-pinene, β-pinene, verbenol, verbenone, verbanone, camphor, fenchone, farnesol, nerolidol, (−)-guaiol, (+)-cedrol, (−)-α-bisabolol, bisabolene, azulenes, (+)-longifolene, (−)-isolongifolol, β-caryphyllene, (+)-aromadendrene, (+)-β-cedrene, phytol, squalene, (+)-limonene, (+)-carvone, (+)-neomenthol, β-caryophyllene oxide, (+)-cedryl acetate; pyrrolidones such as 2-pyrrolidone, 1-methyl-2-pyrrolidone, 1-ethyl-2-pyrrolidone, 1-butyl-2-pyrrolidone, 1-hexyl-2-pyrrolidone, 1-octyl-2-pyrrolidone, 1-(2-hydroxyethyl)-2-pyrrolidone; oxazolidinones such as 4-decyloxazolidin-2-one; substituted amino acetates such as dodecyl-N,N-dimethylaminoacetate, dodecyl-2-methyl-2-(N,N-dimethylaminoacetate); azone and derivatives thereof; surfactants such as sodium lauryl sulphate, cetryltrimethyl ammonium bromide, nonoxynol surfactants, dodecyl betaine, sorbitan monolaureate, polysorbates (e.g. 20, 40, 60, 65, 80), dodecyldimethyl ammoniumpropane sulfate; N,N-dimethyformamide; dimethylsulfoxide, decylmethylsulfoxide; phospholipids such as phosphatidyl glycerol derivatives; cyclodextrin and cyclodextrin complexes; amino acid derivatives such as esters; glucosamine; urea and derivatives; polysaccharides, capsaicin; α-tocopherol; liposomes; invasomes, cyclodextrins such as α-, β- and γ-cyclodextrin, methycyclodextrin, hydroxypropyl β-cyclodextrin, dimethyl-β-cyclodextrin; fusidic acid derivatives such as sodium taurodihydrofusidate, sodium glycodihydrofusidate, sodium phosphate-dihydrofusidate; phosphatidylcholine and homologs, didecanoyl-L-α-phosphatidylcholine; bile salts such as sodium cholate, sodium deoxycholate, sodium glycholate, sodium taurocholate, sodium taurodeoxycholate, sodium glycodeoxycholate; starch, degradable starch, soluble starch; dextrane; cellulose; hyaluronic acid esters, as well as mixtures thereof.

As such, Embodiment 1 is directed to the use of at least one permeation enhancer according to (b) for increasing the oral, rectal, transdermal and/or nasal bioavailability of compounds according to Formula (I), and to the use of at least one permeation enhancer according to (b) in the pharmaceutical composition according to the Embodiment 1.

Compounds according to Formula (I) may be synthesized according to established procedures in the art which may be derived from the experimental part of EP-A1-1762569.

Compounds according to Formula (I) represent pharmaceutically active agents. The term “pharmaceutically active agent” has the same meaning as the terms “drug”, “pharmaceutically active ingredient”, and “pharmaceutically active compound”.

The term “bioavailability” is used herein in accordance with the general knowledge of the person skilled in the art. It indicates the fraction of an (orally) administered dose that reaches the systemic circulation as intact drug, taking into account both absorption and local metabolic degradation (in the case of peroral or rectal administration also the first pass effect is taken into account). [H. P. Rang H, M. M. Dale, J. M. Ritter, R. J. Flower. In: Pharmacology, 6^{th} Edition. Churchill Livingstone, 2007, 106].

The term “absorption” is defined as the passage of a drug and active agent, respectively, from its site of administration into the plasma. It is therefore important for all routes of administration, except intravenous injection [H. P. Rang H, M. M. Dale, J. M. Ritter, R. J. Flower. In: Pharmacology, 6^{th} Edition. Churchill Livingstone, 2007, 104].

The term “permeation enhancer” as used herein refers to natural or synthetic chemical compounds or a combination of natural and/or synthetic chemical compounds that are able to improve/increase/facilitate the absorption and bioavailability, respectively, of a given pharmaceutically active agent or combination of pharmaceutical active agents. The permeation enhancer may also be designated as “absorption” enhancer.

The term “oral administration” as used herein refers to the oral administration of the composition according to Embodiment 1. It includes all kinds of oral administrations such as transmucosal administration, including sublingual administration (administration from below the tongue and absorption via the oral mucosa and the tongue mucosa) and buccal administration (administration through tissues of the buccal vestibule), and peroral administration of the pharmaceutically active compound, i.e. administration by swallowing the composition of Embodiment 1 absorption of the pharmaceutically active agent via the intestinal mucosa.

The term “rectal administration” as used herein refers to the rectal administration of the composition according to Embodiment 1 and the absorption of the pharmaceutically active agent via the intestinal mucosa.

The term “transdermal administration” as used herein refers to the transdermal administration of the composition according to Embodiment 1 and the absorption of the pharmaceutically active agent via the skin (by percutaneous absorption) reaching systemic circulation.

The term “nasal administration” as used herein refers to the nasal administration of the composition according to Embodiment 1 and the absorption of the pharmaceutically active agent via the nasal mucosa reaching systemic circulation and/or olfactory transfer to the central nervous system.

Due to the amino acid in 6-position of the morphinan skeleton of Formula (I) (6-(amino acid)-morphinans), compounds of Formula (I) have an extremely high hydrophilicity which is in particular a result of the zwitterionic moiety derived from the 6-amino acid substituent. In consequence, the bioavailability of these compounds is significantly reduced compared to other morphine-like active agents, such as morphine, oxymorphone, hydromorphone and oxycodon when being administered orally, rectally, transdermally or nasally. Peroral and oral administration is the preferred administration route according to the invention.

Herein, it was surprisingly found that permeation enhancers in accordance with component (b) may be used in order to increase the oral (peroral and transmucosal), rectal, transdermal, and nasal bioavailability of compounds according to Formula (I) (component (a)), with the result that the absorption of these pharmaceutically active substances is significantly improved and/or accelerated upon oral, rectal, transdermal and/or nasal administration.

As regards the peroral and rectal administration of the composition according to Embodiment 1, with peroral administration being particularly preferred, the absorption of the pharmaceutically active compound according to Formula (I) (component (a)) takes place via the intestinal mucosa. The absorption via the intestinal mucosa is improved due to the presence of the at least one permeation enhancer according to (b) in the composition according to Embodiment 1; in case of the transmucosal administration of the composition according to Embodiment 1, the absorption of the pharmaceutically active compound according to Formula (I) (component (a)) occurs via the oral mucosa. The transmucosal administration of the composition according to Embodiment 1 improves the absorption of the pharmaceutically active compound according to Formula (I) via the oral mucosa due to the presence of the at least one permeation enhancer according to (b).

The transdermal administration of the composition according to Embodiment 1, the absorption of the pharmaceutically active compound according to Formula (I) (component (a)) takes place via the skin. The absorption via the skin is improved due to the presence of the at least one permeation enhancer according to (b) in the composition according to Embodiment 1.

As regards the intranasal administration of the composition according to Embodiment 1, the absorption of the pharmaceutically active compound according to Formula (I) (component (a)) takes place via the nasal mucosa. The absorption via the nasal mucosa is improved due to the presence of the at least one permeation enhancer according to (b) in the composition according to Embodiment 1; in case of the intranasal administration of the composition according to Embodiment 1, the absorption of the pharmaceutically active compound according to Formula (I) (component (a)) can occur also via transport across the olfactory membrane directly into the central nervous system. The intranasal administration of the composition according to Embodiment 1 improves the absorption of the pharmaceutically active compound according to Formula (I) via the olfactory membrane due to the presence of the at least one permeation enhancer according to (b).

The enhanced oral, rectal, transdermal and nasal bioavailability of 6-(amino acid)-morphinan derivatives according to Formula (I) in the composition according to Embodiment 1 allows a reduced administration frequency and/or a reduced dosage of the drug upon administration which significantly contributes to patient compliance and patient satisfaction.

In a preferred embodiment of Embodiment 1, which may be combined with any of the embodiments described herein, the composition according to Embodiment 1 is used in an orally, rectally or nasally administered medicament. In a more preferred embodiment of Embodiment 1, the composition according to present invention is used in an orally or rectally administered medicament.

The dotted line between carbon atoms 7 and 8 of Formula (I) designates that these carbon atoms may be unsaturated (olefinic C—C double bond between C7 and C8) or saturated (C—C single bond between C7 and C8).

Some compounds according to Formula (I) may exist in different stereochemical configurations and/or may show more than one crystalline structure, in particular the compounds possessing one or more chiral carbon atom. Embodiment 1 comprises all those specific embodiments, such as diastereomers, enantiomers, polymorphs, in any given or desired mixture or in isolated form.

The nitrogen of Formula (I), to which R₁ is attached, may also be substituted by two substituents R₁, which may be the same or different and which are defined as above, and wherein the second, quarternised substituent R₁ may additionally be hydroxyl, oxyl (N-oxide) and alkoxyl.

The terms “alkyl”, “alkenyl” and “alkynyl” as used herein include both branched and unbranched alkyl, alkenyl and alkynyl groups as well as mono-, di- and trihydroxy-substituted branched and unbranched alkyl, alkenyl and alkynyl groups. These groups furthermore may be substituted once, twice or three times with substituents selected independently from hydroxy, halogen, nitro, cyano, thiocyanato, trifluoromethyl, C1-C₃-alkyl, C₁-C₃-alkoxy, CO₂H, CONH₂, CO₂(C₁-C₃-alkyl), CONH(C₁-C₃-alkyl), CON(C₁-C₃-alkyl)₂, CO(C₁-C₃-alkyl); amino; (C₁-C₃-monoalkyl)amino, (C₁-C₃-dialkyl)amino, C₅-C₆-cycloalkylamino; (C₁-C₃-alkanoyl)amido, SH, SO₃H, SO₃(C₁-C₃-alkyl), SO₂(C₁-C₃-alkyl), SO(C₁-C₃-alkyl), C₁-C₃-alkylthio or C₁-C₃-alkanoylthio. Further suitable substituents are cyclic groups, including carbocycles and heterocycles which may be saturated, unsaturated or aromatic. Preferred examples comprise from 3 to 8 ring atoms, selected from C, N, O, and S. Aryl can be unsubstituted or mono-, di- or tri-substituted, wherein the substituents can be chosen independently from hydroxy, halogen, nitro, cyano, thiocyanato, trifluoromethyl, C₁-C₃-alkyl, C₁-C₃-alkoxy, CO₂H, CONH₂, CO₂(C₁-C₃-alkyl), CONH(C₁-C₃-alkyl), CON(C₁-C₃-alkyl)₂, CO(C₁-C₃-alkyl); amino; (C₁-C₃-monoalkyl)amino, (C₁-C₃-dialkyl)amino, C₅-C₆-cycloalkylamino; (C₁-C₃-alkanoyl)amido, SH, SO₃H, SO₃(C₁-C₃-alkyl), SO₂(C₁-C₃-alkyl), SO(C₁-C₃-alkyl), C₁-C₃-alkylthio or C₁-C₃-alkanoylthio. Further suitable substituents are cyclic groups, including carbocycles and heterocycles which may be saturated unsaturated or aromatic. Preferred examples comprise from 3 to 8 ring atoms, selected from C, N, O, and S.

The term “aryl” as used herein defines aromatic rings comprising preferably from 5 to 14 ring atoms and the term aryl comprises furthermore carboxylic aryl groups as well as heterocyclic aryl groups, comprising preferably from 1 to 3 heteroatoms, selected from N, O and S. The aryl groups as defined herein may furthermore be fused ring systems such as naphthyl or anthracenyl or the corresponding heterocyclic groups comprising from 1 to 3 heteroatoms selected from N, O, and S.

The above definitions for “alkyl”, “alkenyl”, “alkynyl” and “aryl” are valid for all embodiments of Embodiment 1.

In a preferred embodiment, the composition according to Embodiment 1 comprises at least one compound of formula (I) wherein the substituents R₁, R₂, R₃, R₄, R₅ and R₆ have the following meaning:

R₁ is selected from hydrogen; C₁-C₁₂-alkyl; C₂-C₁₂-alkenyl; C₂-C₁₂-alkynyl; C₁-C₁₂-monohydroxyalkyl; C₂-C₁₂-dihydroxyalkyl; C₃-C₁₂-trihydroxyalkyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

R₂ is selected from hydrogen; C₁-C₁₂-alkyl; C₁-C₁₂-monohydroxyalkyl; C₂-C₁₂-dihydroxyalkyl; C₃-C₁₂-trihydroxyalkyl; C₂-C₁₂-alkenyl; C₂-C₁₂-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

R₃ is selected from hydrogen; C₁-C₁₂-alkyl; C₂-C₁₂-alkenyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; alkoxyalkyl, wherein alkoxy is C₁-C₆-alkoxy and alkyl is C₁-C₆-alkyl;

R₄ is selected from hydrogen; C₁-C₁₂-alkyl; C₂-C₁₂-alkenyl; C₂-C₁₂-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl; C₂-C₁₂-alkanoyl; C₃-C₁₂-alkenoyl; C₃-C₁₂-alkinoyl; C₇-C₁₆-arylalkanoyl, wherein aryl preferably is C₆-C₁₀-aryl and alkanoyl preferably is C₁-C₆-alkanoyl; C₉-C₁₆-arylalkenoyl, wherein aryl preferably is C₆-C₁₀-aryl and alkenoyl preferably is C₃-C₆-alkenoyl; C₉-C₁₆-arylalkinoyl, wherein aryl preferably is C₆-C₁₀-aryl and alkinoyl preferably is C₃-C₆-alkinoyl;

R₅ is selected from hydrogen, C₁-C₁₂-alkyl; C₂-C₁₂-alkenyl; C₂-C₁₂-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

CH(A)CO₂B, wherein A is selected from hydrogen; hydroxyl; C₁-C₁₂-alkyl; C₂-C₁₂-alkenyl; C₂-C₁₂-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl; amino; C₁-C₆-alkylamino; guanidino; C₁-C₆-alkyl-CO₂B; C₁-C₆-monohydroxyalkyl; C₂-C₆-dihydroxyalkyl; C₃-C₆-trihydroxyalkyl; C₁-C₆-monoaminoalkyl; C₂-C₆-diaminoalkyl; C₃-C₆-triaminoalkyl; C₁-C₆-alkylguanidino; C₁-C₆-alkylcarboxamide; C₁-C₆-alkylhydroxycarbonyl; C₁-C₆-sulfhydrylalkyl; C₂-C₁₂-alkylthioalkyl, wherein alkylthio is preferably C₁-C₆ and alkyl is preferably C₁-C₆; and wherein B is selected from hydrogen; C₁-C₁₂-alkyl; C₂-C₁₂-alkenyl; C₂-C₁₂-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl; phenyl; substituted phenyl; (C₁-C₆-alkyl)CONH₂;

CH(A)SO₃B, wherein A and B are defined as above;

CH(A)COB1, wherein A is defined as above, and B1 is NH₂; NH—C1-C12, more preferably NH—C₁-C₆-alkyl; N—(C1-C12)₂, more preferably N—(C₁-C₆)₂-alkyl;

R₆ is CH(A)CO₂H, wherein A is selected from hydrogen; hydroxyl; C₁-C₁₂-alkyl; C₂-C₁₂-alkenyl; C₂-C₁₂-alkynyl; C₇-C₁₆-arylalkyl, wherein aryl preferably is C₆-C₁₀-aryl and alkyl preferably is C₁-C₆-alkyl; C₃-C₁₀-cycloalkyl; C₄-C₁₆-cycloalkylalkyl, wherein cycloalkyl preferably is C₃-C₁₀-cycloalkyl and alkyl preferably is C₁-C₆-alkyl; C₁-C₆-monohydroxyalkyl; C₂-C₆-dihydroxyalkyl; C₃-C₆-trihydroxyalkyl; C₁-C₆-monoaminoalkyl; C₂-C₆-diaminoalkyl; C₃-C₆-triaminoalkyl; C₁-C₆-alkylguanidino; C₁-C₆-alkylcarboxamide; C₁-C₆-alkylhydroxycarbonyl; C₁-C₆-sulfhydrylalkyl; C₂-C₁₂-alkylthioalkyl, wherein alkylthio is preferably C₁-C₆ and alkyl is preferably C₁-C₆;

or a pharmaceutically and pharmacologically acceptable acid addition salt or base addition salt thereof.

In an alternative preferred embodiment of the composition according to Embodiment 1, the substituents R₁, R₂, R₃, R₄, R₅ and R₆ in the compound of Formula (I) are defined as above, with the difference that R₂ is not hydrogen.

In a more preferred embodiment, the composition according to embodiment 1 comprises at least one compound of formula I wherein the substituents R₁, R₂, R₃, R₄, R₅, and R₆ have the following meaning:

R₁ is selected from hydrogen; C₁-C₆-alkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

R₂ is selected from hydrogen, C₁-C₆-alkyl; C₁-C₆-monohydroxyalkyl; C₂-C₆-dihydroxyalkyl; C₃-C₆-trihydroxyalkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

R₃ is selected from hydrogen; C₁-C₆-alkyl; C₂-C₆-alkenyl; C₂-C₆-alkynyl, C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl;

R₄ is selected from hydrogen; C₁-C₆-alkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

R₅ is selected from hydrogen, C₁-C₆-alkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

CH(A)CO₂B, wherein A is selected from hydrogen; C₁-C₆-alkyl; C₇-C₁₆-arylalkyl, wherein aryl preferably is C₆-C₁₀-aryl and alkyl preferably is C₁-C₆-alkyl; C₃-C₁₀-cycloalkyl; C₄-C₁₆-cycloalkylalkyl, wherein cycloalkyl preferably is C₃-C₁₀-cycloalkyl and alkyl preferably is C₁-C₆-alkyl; and wherein B is selected from hydrogen; C₁-C₆-alkyl; C₂-C₆-alkenyl; C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl; phenyl; substituted phenyl; (C₁-C₆-alkyl)CONH₂;

CH(A)SO₃B, wherein A and B are defined as above;

CH(A)COB1, wherein A is defined as above, and B1 is NH₂;

R₆ is CH(A)CO₂H, wherein A is selected from hydrogen; C₁-C₆-alkyl; C₇-C₁₆-arylalkyl, wherein aryl preferably is C₆-C₁₀-aryl and alkyl preferably is C₁-C₆-alkyl; C₃-C₁₀-cycloalkyl; C₄-C₁₆-cycloalkylalkyl, wherein cycloalkyl preferably is C₃-C₁₀-cycloalkyl and alkyl preferably is C₁-C₆-alkyl;

or a pharmaceutically and pharmacologically acceptable acid addition salt or base addition salt thereof.

In an alternative more preferred embodiment of the composition according to Embodiment 1, the substituents R₁, R₂, R₃, R₄, R₅ and R₆ in the compound of Formula (I) are defined as above, with the difference that R₂ is not hydrogen.

In an even more preferred embodiment, the composition according to embodiment 1 comprises at least one compound of Formula (I) wherein the substituents R₁, R₂, R₃, R₄, R₅, and R₆ have the following meaning:

R₁ is selected from hydrogen; C₁-C₆-alkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₇-C₁₆-arylalkyl, wherein aryl preferably is C₆-C₁₀-aryl and alkyl preferably is C₁-C₆-alkyl;

R₂ is selected from hydrogen, C₁-C₆-alkyl; C₂-C₆-alkenyl; C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

R₃ is selected from hydrogen; C₁-C₆-alkyl; C₇-C₁₆-arylalkyl, wherein aryl preferably is C₆-C₁₀-aryl and alkyl preferably is C₁-C₆-alkyl;

R₄ is selected from hydrogen and C₁-C₆-alkyl, C2-C6-alkynyl;

R₅ is selected from hydrogen, C₁-C₆-alkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl; CH(A)CO₂B, wherein A is selected from hydrogen; C₁-C₆-alkyl; C₇-C₁₆-arylalkyl, wherein aryl preferably is C₆-C₁₀-aryl and alkyl preferably is C₁-C₆-alkyl; and wherein B is selected from hydrogen; C₁-C₆-alkyl; C₂-C₆-alkenyl; C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl; phenyl; substituted phenyl;

R₆ is CH(A)CO₂H, wherein A is selected from hydrogen; C₁-C₆-alkyl; C₇-C₁₆-arylalkyl, wherein aryl preferably is C₆-C₁₀-aryl and alkyl preferably is C₁-C₆-alkyl; C₄-C₁₆-cycloalkylalkyl, wherein cycloalkyl preferably is C₃-C₁₀-cycloalkyl and alkyl preferably is C₁-C₆-alkyl;

or a pharmaceutically and pharmacologically acceptable acid addition salt or base addition salt thereof.

In an alternative even more preferred embodiment of the composition according to Embodiment 1, the substituents R₁, R₂, R₃, R₄, R₅ and R₆ in the compound of Formula (I) are defined as above, with the difference that R₂ is not hydrogen.

Further preferred embodiments of compounds according to Formula (I) (component (a)), which may be combined with any of the preceding or following embodiments of the present

Embodiment 1, are shown in the following Table 1-1.

TABLE 1-1
Further preferred embodiments of component (a) (compounds according
to Formula (I))

| | Further preferred | Particularly preferred | Most preferred |
|---|---|---|---|
| R₁ | C1-C6-alkyl, | methyl, ethyl, 2- | methyl, |
| | C1-C6-alkenyl, C₄-C₁₆- | phenylethyl, | cyclopropylmethyl, or |
| | cycloalkylalkyl, or C₇- | cyclopropylmethyl or | allyl |
| | C₁₆-arylalkyl | allyl | |
| R₂ | as defined above for | C1-C6-alkyl, | Methyl, ethyl, benzyl |
| | the even more | particularly methyl, | or |
| | preferred embodiment | ethyl and (substituted) | 3-phenylpropyl |
| | or the alternative even | propyl; arylalkyl as | |
| | more preferred | defined above for the | |
| | embodiment, with the | even more preferred | |
| | preference that R₂ is | embodiment | |
| | not a group which | | |
| | forms an ester with O | | |
| R₃ | hydrogen, | hydrogen, methyl, and | hydrogen |
| | C1-C6-alkyl or C7- | benzyl | |
| | C16-arylalkyl | | |
| R₄ | hydrogen, | hydrogen, methyl or | hydrogen |
| | C1-C6-alkyl or C2-C6- | propargyl | |
| | alkynyl | | |
| R₅ | as defined above for | hydrogen, C1 -C6 alkyl, | hydrogen |
| | the even more | C₇-C₁₆-arylalkyl, C₄- | |
| | preferred embodiment | C₁₆-cycloalkylalkyl | |
| R₆ | as defined above for | CH(A)CO₂H, wherein A | CH(A)CO₂H, wherein |
| | the even more | is hydrogen, C1-C6- | A is hydrogen, C1- |
| | preferred embodiment | alkyl, C4-C16- | C6-alkyl, C7-C16- |
| | | cycloalkylalkyl, C7- | arylalkyl |
| | | C16-arylalkyl | |
| Configuration | α, β | α, β | α, β |
| C6 | | | |
| Connectivity | C—C single bond, | C—C single bond | C—C single bond |
| C7-C8 | C—C double bond | | |

All preferred embodiments for compounds according to Formula (I) (component (a)) as reflected in Table 1-1 may be combined with one another as well as with all the preceding and following embodiments of Embodiment 1. Also all alternative embodiments for compounds according to Formula (I) may be combined with one another as well as with all the preceding and following embodiments of Embodiment 1.

In a particularly preferred embodiment of Embodiment 1, R₁ and R₂ of compounds according to Formula (I) represent alkyl at the same time, in particular methyl. In an alternative particularly preferred embodiment, R₁ represents cycloalkylalkyl, in particular cyclopropylmethyl, and R₂ represents alkyl or arylalkyl, in particular methyl, benzyl or 3-phenylpropyl. 6-(Amino acid)-morphinan derivatives with this substitution pattern have been found to be particularly suitable for the enhancement of the oral, rectal, transdermal and nasal bioavailability in combination with at least one permeation enhancer according to (b).

As noted above, compounds according to Formula (I) (component (a)) may also be in the form of pharmaceutically and pharmacologically acceptable salts, which may be derived from acid addition or base addition to compounds of Formula (I). Both inorganic and organic salts are suitable. Examples of suitable inorganic salts are hydrochlorides, hydrobromides, hydroiodides, sulphates, phosphates and tetrafluoroborates. Examples for suitable organic salts are acetates, tartrates, lactates, benzoates, stearates, palmoates, methane sulphonates, salicylates, fumarates, maleinates, succinates, aspartates, citrates, oxalates, trifluoroacetates and orotates.

In a preferred embodiment of Embodiment 1, which may be combined with any of the preceding or following embodiments, the acid addition salt of a compound according to Formula (I) (component (a)) is the hydrochloride, the hydrobromide, the tetrafluoroborate or the trifluoroacetate. Particularly preferred are the hydrochloride, the tetrafluoroborate and the trifluoroacetate.

In an alternative preferred embodiment of Embodiment 1, which may be combined with any of the preceding or following embodiments, the compound according to Formula (I) (component (a)) is in the form of a base addition salt. Suitable base addition salts of compounds according to Formula (I) (component (a)) include for example metal salts, such as lithium salts, sodium salts, potassium salts, beryllium salts, magnesium salts, calcium salts, strontium salts, aluminum salts and zinc salts; ammonium salts, such as C₁-C₃₀ monoalkylammonium salts, C₁-C₃₀ dialkylammonium salts, C₁-C₃₀ trialkylammonium salts, C₁-C₃₀ tetraalkylammonium salts; C₂-C₃₀ monoalkenylammonium salts, C₂-C₃₀ dialkenylammonium salts, C₂-C₃₀ trialkenylammonium salts, C₂-C₃₀ tetraalkenylammonium salts; C₂-C₃₀ monoalkynylammonium salts, C₂-C₃₀ dialkynylammonium salts, C₂-C₃₀ trialkynylammonium salts, C₂-C₃₀ tetraalkynylammonium salts; C₄-C₃₀ mono(cycloalkylalkylammonium) salts, C₄-C₃₀ di(cycloalkylalkylammonium) salts, C₄-C₃₀ tri(cycloalkylalkylammonium) salts, C₄-C₃₀ tetra(cycloalkylalkylammonium) salts, wherein cycloalkyl is C₃-C₁₀-cycloalkyl and alkyl is C₁-C₂₇-alkyl; C₅-C₃₀ mono(cycloalkylalkenylammonium) salts, C₅-C₃₀ di(cycloalkylalkenylammonium) salts, C₅-C₃₀ tri(cycloalkylalkenylammonium) salts, C₅-C₃₀ tetra(cycloalkylalkenylammonium) salts, wherein cycloalkyl is C₃-C₁₀-cycloalkyl and alkenyl is C₂-C₂₇-alkenyl; C₅-C₃₀ mono(cycloalkylalkynylammonium) salts, C₅-C₃₀ di(cycloalkylalkynylammonium) salts, C₅-C₃₀ tri(cycloalkylalkynylammonium) salts, C₅-C₃₀ tetra(cycloalkylalkynylammonium) salts, wherein cycloalkyl is C₃-C₁₀-cycloalkyl and alkynyl is C₂-C₂₇-alkynyl; C₇-C₃₀ mono(arylalkylammonium) salts, C₇-C₃₀ di(arylalkylammonium) salts, C₇-C₃₀ tri(arylalkylammonium) salts, C₇-C₃₀ tetra(arylalkylammonium) salts, wherein aryl is C₆-C₁₀-aryl and alkyl is C₁-C₂₄-alkyl; C₈-C₃₀ mono(arylalkenylammonium) salts, C₈-C₃₀ di(arylalkenylammonium) salts, C₈-C₃₀ tri(arylalkenylammonium) salts, C₈-C₃₀ tetra(arylalkenylammonium) salts, wherein aryl is C₆-C₁₀-aryl and alkenyl is C₂-C₂₄-alkenyl; C₈-C₃₀ mono(arylalkynylammonium) salts, C₈-C₃₀ di(arylalkynylammonium) salts, C₈-C₃₀ tri(arylalkynylammonium) salts, C₈-C₃₀ tetra(arylalkynylammonium) salts, wherein aryl is C₆-C₁₀-aryl and alkynyl is C₂-C₂₄-alkynyl, combinations of the ammonium salts listed above, and salts derived from heterocyclic bases, in particular heterocyclic nitrogen bases. These include salts derived from heterocyclic compounds comprising the following cycles: pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, pyrrolidine, imidazolidine, pyrazolidine, oxazolidine, isoxazolidine, thiazolidine, isothiazolidine, thiadiazolidine, sulpholane, imidazolidine, pyridine, pyridazine, pyrazine, pyrimidine, piperazine, piperidine, morpholine, tetrazole, triazole, triazolidine, tetrazolidine, azepine, homopiperazine and azetidine.

In a more preferred embodiment, which may be combined with any of the preceding and following embodiments of Embodiment 1, the base addition salt of component (a) is selected from the group consisting of metal and ammonium salts. Particularly preferred are the lithium, sodium, potassium, magnesium and calcium salts.

The compounds according to Formula (I) (component (a)) also include alcoholates and hydrates. The term “hydrate” as used herein refers to a pharmaceutically and pharmacologically acceptable salt of component (a) including water of crystallization. The term “alcoholate” as used herein refers to a pharmaceutically and pharmacologically acceptable salt of component (a) as described above wherein alcohol, such as ethanol, takes the place of water of crystallization or coexists besides water of crystallization.

Compounds according to Formula (I) may be synthesized according to established procedures in the art which may be derived from the experimental part of EP-A1-1762569, under due consideration of the synthetic methods disclosed by Parra et al., Eur. J. Org. Chem. 2003, 1386-1388.

In Embodiment 1, the at least one permeation enhancer according to (b) is used to improve and accelerate the oral, rectal, transdermal, nasal absorption of compounds according to Formula (I).

In a preferred embodiment, which may be combined with any of the preceding or following embodiment, Component (b) of the composition according to Embodiment 1 is selected from the group consisting of saturated and/or unsaturated organic fatty acids, or pharmaceutically and pharmacologically acceptable salts thereof, and thiomers.

The term “saturated and/or unsaturated organic fatty acid” as used herein refers to a carboxylic acid consisting of at least one carboxylic group and a linear or non-linear aliphatic tail consisting of carbon atoms. The term “saturated” indicates that the aliphatic tail chain is made of C—C-single bonds. The term “unsaturated” means that the aliphatic tail chain comprises at least one olefinic bond. An “olefinic bond” means a C—C-double bond which may be cis- or trans-configurated. The organic fatty acid of Embodiment 1 comprises preferably 4 to 22 carbon bonds, more preferably 6 to 20 carbon atoms, and particularly preferably 8 to 18 carbon atoms.

Examples for saturated organic fatty acids bearing a terminal carboxylic group and a saturated, linear alkyl chain include behenic acid (C22), arachidic acid (C20), stearic acid (C18), palmitic acid (C16), myristic acid (C14), lauric acid (C12), capric acid (C10), and caprylic acid (C8).

Examples for unsaturated organic fatty acids with one terminal carboxylic group and a linear alkyl at least one olefinic bond include erucic acid (C22, cis-Δ¹³), eicosapentaenoic acid (C20, cis,cis,cis,cis,cis-Δ⁵,Δ⁸,Δ¹¹,Δ¹⁴,Δ¹⁷), arachidonic acid (C20, cis,cis,cis,cis-Δ⁵Δ⁸,Δ¹¹,Δ¹⁴), α-linolenic acid (C18, cis,cis,cis-Δ⁹,Δ¹²,Δ¹⁵), linoelaidic acid (C18, trans,trans-Δ⁹,Δ¹²), linoleic acid (C18, cis,cis-Δ⁹,Δ¹²), vaccenic acid (C18, trans-Δ¹¹), elaidic acid (C18, trans-Δ⁹), oleic acid (C18, cis-Δ⁹), sapienic acid (C16, cis-Δ⁶), palmitoleic acid (C16, cis-Δ⁹) myristoleic acid (C14, cis-Δ⁹).

The aliphatic tail chain of the organic fatty acid may be further substituted such as by alkyl groups, hydroxyl groups and/or carboxyl groups. Examples for saturated and unsaturated fatty acids wherein the aliphatic tail chain bears at least one further substituent are ricinoleic acid and phytanic acid.

The substituents of the aliphatic tail chain of the organic fatty acid may also be further substituted. In particular, hydroxyl groups may be polyalkoxylated, for example by reaction with ethylene oxide to form polyethylene glycol ethers resulting in polyalkoxylated saturated and/or unsaturated organic fatty acids. In a preferred embodiment of the composition of Embodiment 1, which may be combined with any of the preceding and following embodiments, the at least one permeation enhancer (component (b)) is a polyalkoxylated saturated and/or unsaturated organic fatty acid. Particularly preferred in this regard is Cremophor® EL Castor Oil by BASF, wherein the major component is polyethoxylated castor oil, which is derived by reacting castor oil with ethylene oxide in a molar ratio of 1:35.

A pharmaceutically and pharmacologically acceptable salt of the saturated and/or unsaturated organic fatty acid may be derived from acid or base addition to the fatty acid. In a preferred embodiment of Embodiment 1, which may be combined with any of the preceding or following embodiments, the pharmaceutically and pharmacologically acceptable salt of the saturated and/or unsaturated organic fatty acid is a metal base addition salt, such as a lithium salt, sodium salt, potassium salt, beryllium salt, magnesium salt, and calcium salt. Particularly preferred is a sodium or potassium salt.

The term “thiomer” as used herein refers to thiolated polymers, which display thiol bearing side chains and possess mucoadhesive properties. The mechanism responsible for the permeation enhancing effect seems to be based on the thiol groups of the polymer which can form disulfide bonds between thiolated polymers and cysteine-rich subdomains of mucus glycoproteins. Cationic thiomers (e.g. chitosan-cysteine, chitosan-thiobutylamidine, chitosan-thioglycolic acid) and anionic thiomers (e.g. polyacrylic acid-cysteine, polyacrylic acid-cysteamine, carboxymethylcellulose-cysteine, alginate-cysteine) are known. Thiomers are effective carriers for the delivery of therapeutics (A. Bernkop-Schnürch, C. E. Kast, D. Guggi, J. Control. Release 2005, 93, 95-103; A. Bernkop-Schnurch, Adv. Drug. Deliv. Rev. 2005, 57, 1569-82; Rakesh Kumar, V. R. Sinha, Reactive and Functional Polymers 2013, 73, 1156-66). Herein, the thiomer is preferably selected from the group consisting of PAA₄₅₀ and PAA₄₅₀-Cys.

In a particularly preferred embodiment of Embodiment 1, which may be combined with any of the preceding or following embodiments, the at least one permeation enhancer (component (b)) is selected from the group consisting of capric acid, or a pharmaceutically and pharmacologically acceptable salt thereof, lauric acid, or a pharmaceutically and pharmacologically acceptable salt thereof, Cremophor® EL, PAA₄₅₀ and PAA₄₅₀-Cys.

Preferred as permeation enhancers are mixtures of hydrophilic and hydrophobic compounds, resulting in an amphiphilic composition. Examples thereof include mixtures of glycols and/or glycerol (and derivatives thereof) with fatty acid esters of glycols (such as polyethylene glycol) and/or esters of glycerols (such as glycerol polyethylene glycol ticinoleate). A prominent and commercially available example thereof is Cremophor® EL (sold also as Kolliphor™ EL).

These compounds are particularly suitable to act as permeation enhancers and to increase the oral and rectal bioavailability of compounds according to Formula (I) in accordance with the teaching of Embodiment 1.

Also particularly preferred, in combination with any of the preceding or following embodiments of Embodiment 1, is a composition comprising at least one compound according to Formula (I) (component (a)) wherein the substituents have the following meaning: R₁ is methyl, cyclopropylmethyl, or allyl; R₂ is methyl, or 3-phenylpropyl; R₃ is hydrogen; R₄ is hydrogen; R₅ or R₆ is CH₂CO₂H; R₅ or R₆ is hydrogen; the configuration of C6 is α or β, preferably β; C7 and C8 are connected via a single bond; or a pharmaceutically and pharmacologically acceptable acid addition salt or base addition salt thereof; and at least one permeation enhancer (component (b)) to increase the oral and rectal bioavailability of the at least one compound of Formula (I), selected from the group consisting of capric acid, or a pharmaceutically and pharmacologically acceptable salt thereof, lauric acid, or a pharmaceutically and pharmacologically acceptable salt thereof, Cremophor® EL, PAA₄₅₀ and PAA₄₅₀-Cys.

In this particularly preferred embodiment, the oral, rectal, transdermal and nasal, particularly the oral and rectal bioavailability of the active component (a) is particularly improved and accelerated with the result that its absorption is improved and increased. In consequence, pharmaceutical compositions according to Embodiment 1 comprising these particular components (a) and (b) are particularly suitable for reducing the administration frequency of the drug, reducing the dosage of the drug and increasing patient compliance and patient satisfaction.

Permeation enhancers according to component (b), which are particularly suitable for transdermal administration of the composition of Embodiment 1 are selected from acetone; alcohols, glycols and glycerides such as ethanol, caprylic alcohol, propylene glycol; essential oils such as niaouli oil, eucalyptus oil, Alpinia oxyphylla oil, turpentine oil, sweet basil oil, tulsi oil, cardamom oil, peppermint oil, fennel oil, black cumin oil; terpenes such as geraniol, nerol, linalool, limonene, α-terpineol, β-terpineol, γ-terpineol, menthol, carveol, menthone, pulegone, iso-pulegone, piperitone, carvomenthone, carvone, 1,8-cineole, α-thujene, car-3-ene, α-pinene, 3-pinene, verbenol, verbenone, verbanone, camphor, fenchone, farnesol, nerolidol, (−)-guaiol, (+)-cedrol, (−)-α-bisabolol, bisabolene, azulenes, (+)-longifolene, (−)-isolongifolol, β-caryphyllene, (+)-aromadendrene, (+)-β-cedrene, phytol, squalene, (+)-limonene, (+)-carvone, (+)-neomenthol, β-caryophyllene oxide, (+)-cedryl acetate; pyrrolidones such as 2-pyrrolidone, 1-methyl-2-pyrrolidone, 1-ethyl-2-pyrrolidone, 1-butyl-2-pyrrolidone, 1-hexyl-2-pyrrolidone, 1-octyl-2-pyrrolidone, 1-(2-hydroxyethyl)-2-pyrrolidone; oxazolidinones such as 4-decyloxazolidin-2-one; substituted amino acetates such as dodecyl-N,N-dimethylaminoacetate, dodecyl-2-methyl-2-(N,N-dimethylaminoacetate); azone and derivatives thereof; surfactants such as sodium lauryl sulphate, cetryltrimethyl ammonium bromide, nonoxynol surfactants, dodecyl betaine, sorbitan monolaureate, polysorbates (e.g. 20, 40, 60, 65, 80), dodecyldimethyl ammoniumpropane sulfate; N,N-dimethyformamide; dimethylsulfoxide, decylmethylsulfoxide; phospholipids such as phosphatidyl glycerol derivatives; cyclodextrin and cyclodextrin complexes; amino acid derivatives such as esters; glucosamine; urea and derivatives; polysaccharides, capsaicin; α-tocopherol; liposomes; invasomes.

Permeation enhancers according to component (b), which are particularly suitable for transdermal administration of the composition of Embodiment 1 are selected from cyclodextrins such as α-, β- and γ-cyclodextrin, methycyclodextrin, hydroxypropyl β-cyclodextrin, dimethyl-β-cyclodextrin; fusidic acid derivatives such as sodium taurodihydrofusidate, sodium glycodihydrofusidate, sodium phosphate-dihydrofusidate; phosphatidylcholine and homologs, didecanoyl-L-α-phosphatidylcholine; bile salts such as sodium cholate, sodium deoxycholate, sodium glycholate, sodium taurocholate, sodium taurodeoxycholate, sodium glycodeoxycholate; starch, degradable starch, soluble starch; dextrane; cellulose; hyaluronic acid esters; mucoadhesive drug delivery systems.

As regards the amounts of components (a) and (b) in the composition according to Embodiment 1, component (a) is preferably comprised in an amount of 0.001 to 98% by weight, based on the total volume of the composition (m/v). More preferably, the amount of component (a) in the composition of Embodiment 1 is 0.01 to 95% by weight, 0.02 to 90% by weight, 0.05 to 80% by weight, 0.06 to 70% by weight, 0.07 to 50% by weight, and 0.09 to 30% by weight, based on the total volume of the composition (m/v). Particularly preferred is an amount of component (a) of 0.1 to 20% by weight, based on the total volume of the composition (m/v). These preferred embodiments are combinable with any of the previous and following embodiments.

The amount of the at least one permeation enhancer (component (b)) in the composition according to Embodiment 1 is preferably, and in combination with any of the preceding and following embodiments, 0.01 to 60% by weight, based on the total volume of the composition (m/v). More preferred is an amount of 0.05 to 40% by weight, and 0.1 to 30% by weight, based on the total volume of the composition (m/v). Particularly preferred is an amount of 0.2 to 20% by weight of component (b), based on the total volume of the composition (m/v).

The composition according to Embodiment 1 comprises in a preferred embodiment, which may be combined with any of the preceding and following embodiments, at least one further pharmaceutically acceptable excipient. Pharmaceutically acceptable excipients are additional materials used in pharmaceutical compositions in order to bind the pharmaceutically active ingredients into a form suitable for administration.

The at least one further pharmaceutically acceptable excipient may be selected from the group consisting of magnesium stearate, glucose, saccharides and their derivatives such as sucrose, lactose; polysaccharides and their derivatives: starches, xanthan gum, cellulose, plant cellulose, microcrystalline cellulose, cellulose ethers such as hydroxypropyl cellulose, hydroxymethyl cellulose, methyl cellulose, hydroxypropyl methylcellulose, hydroxyethyl cellulose, carboxymethyl cellulose; pectins; tragacanth; sugar alcohols such as xylitol, sorbitol, mannitol or maltitol; gelatin; synthetic polymers such as polyvinylpyrrolidones (kollidons), polyethylene oxides, shellac, polyacrylic acids, polyacrylates, gelatinpolysuccinat, polysorbates; water, ethyl alcohol, isopropanol, cetearyl alcohol, glycerol, glycerol-gelatin, glycerol monostearate, propylene glycol, polypropyleneglycols, polyethylene glycols, macrogols, isopropyl myristate, cetyistearyl alcohol, cetyl alcohol, vaselin, cocoa butter, hard fat, witepsols, witocans, imwitors, softigens, dynasans, synthetic fats, solid paraffin, liquid paraffin, glycerinated gelatin, hydrogenated vegetable oils, miglyol neutral oils, miglyol gels, wax esters, dynaserins, softisans, dynacets, nacols, galenols, lipoxols, suppocires, isofols, stearates, polysorbates, chremophor RH 40, chremophor A grades, macrogols; fatty acids, waxes, plastics, plant fibers, Eudragit polymers, Eudragit L, Eudragit S, Eudragit FS 30 D, Eudragit RL, Eudragit RS, Eudragit NE; poly(meth)acrylates, methyl acrylate-methacrylic acid copolymers, methyl methacrylate-methacrylic acid copolymers, cellulose acetate succinate, hydroxypropyl methylcellulose phthalate, hydroxypropyl methylcellulose acetate succinate (hypromellose acetate succinate), polyvinyl acetate phthalate; alginates such as sodium alginate, calcium alginate or potassium alginate; stearic acid, corn protein such as zein, carbopol polymers; crosslinked polymers such as crospovidone or croscarmellose sodium; modified starch such as sodium starch glycolate; dibasic calcium phosphate, calcium carbonate, magnesium carbonate, sodium hydroxide, sodium hydrogen carbonate, sodium hydrogen phosphate, sodium citrate, citric acid, sodium chloride, trometamol, triethanolamine, magnesium oxide, magnesium hydroxide, clay, magnesium aluminum silicate, zinc oxide, zinc sulfate, sodium stearyl fumarate, sodium behenate, sorbitan esters, vegetable oils and fats, lecithin; polaxamers, lutrols, flavourings (natural or artificial) such as mint, cherry, anise, peach, apricot, liquorice, raspberry, vanilla, saccharin sodium, cyclamate, aspartame, sucralose; colours; (lubricants such as) talc, bentonite, silica, colloidal silicon dioxide, fumed silica; (antioxidants) parabens such as methyl paraben or propyl paraben, sodium benzoate, benzyl alcohol, EDTA, butylhydroxytoluene; quaternary ammonium compounds (QUATS) such as benzalkonium chloride, cetrimide, CEDAB or CTAB; all-rac-alpha-tocopheryol, all-rac-alpha-tocopheryl acetate, dexpanthenol; microspheres; liposomes; modified carboxymethyl cellulose; cellulose ethers such as cellulose acetate phthalate; cellulose acetate trimellitate; hydroxypropylmethyl cellulose phthalate; polymethacrylates; methacrylic acid; ethyl acrylate; alginic; fatty acids; waxes; shellac; plastics; hydrocolloids; biogums; plant fibers; and titanium dioxide.

It is particularly preferred to select the at least one additional pharmaceutically acceptable excipient from the group consisting of lactose; polysaccharides and their derivatives (starches, cellulose and derivatives such as ethers and esters); polyvinyl derivatives; polymethacrylates; water; ethyl alcohol; isopropanol; cetearyl alcohol; sorbitol; gelatin; glycol; polyethylene glycol; glycerol; glycerol-gelatin; glycerol monostearate; vaselin; cacao butter; hard fat; synthetic fats; vegetable oils such as soy oil; hydrogenated vegetable oils; vegetable fats; fatty acids; stearic acid; stearates; dexpanthenol; magnesium carbonate; magnesium oxide; magnesium stearate; hydroxypropyl cellulose; hydroxyethyl cellulose; and talc.

The composition according Embodiment 1 may comprise the at least one pharmaceutically acceptable excipient in an amount of 0.1 to 80% by weight, based on the total volume of the composition according to Embodiment 1 (m/v). Preferably, the at least one further pharmaceutically acceptable excipient is comprised in an amount of 0.2 to 70% by weight, and 0.5 to 60% by weight, based on the total volume of the composition of Embodiment 1 (m/v). It is particularly preferred to include the at least one pharmaceutically acceptable excipient in an amount of 1 to 50% by weight, based on the total volume of the composition according to Embodiment 1 (m/v).

The composition according to Embodiment 1 may be produced by blending an effective amount of at least one compound according to Formula (I) (component (a)) with an effective amount of the at least one permeation enhancer according to (b). The mixing of components (a) and (b) is preferably performed at room temperature (20 to 30° C.). Pharmaceutically acceptable excipients as described above may be added in the required amount at any stage, i.e., an effective amount of component (a) or component (b) may be mixed with an effective amount of component (b) or component (a), followed by the addition of an effective amount of at least one pharmaceutically acceptable excipient may added, or an effective amount of component (a) or component (b) may be mixed with an effective amount of at least one pharmaceutically acceptable excipient followed by the addition of an effective amount of component (b) or component (a). Solid components are preferably added as a powder. If necessary, the resulting mixture may be sonicated at 20 to 50° C. for 5 min to 1 h in order to dissolve or disperse the components homogenously. The mixture is then mixed for another 2 to 24 h in order to obtain a homogenous and uniform distribution of components which is preferably done in a roll mill by rolling the mixture.

The composition according to Embodiment 1 may be used for the treatment of pain.

Specifically, the pain which may be treated by the composition of Embodiment 1 comprises acute and chronic pain; pain on the locomotor system such as pain in the neck, back, hip, knee, shoulder; arthritic pain, osteoarthritic pain, or myofacial pain; treatment of complex regional pain syndromes, phantom pain, facial neuralgia, postherpetic neuralgia, rheumatalgia, rheumatic pain, sciatic pain, spinal pain, cancer pain, tumor pain, pain from burns, pain after accidents, pain due to acute and chronic inflammation, visceralgia, headaches such as for example tension headaches, cervically related headache or migraine, pain after central lesions such as for example with paraplegia or thalamic lesions, neuralgic pain such as zoster neuralgia, postzoster neuralgia, ischaemic pain such as angina pectoris or peripheral occlusive arterial disease, postoperative pain, neuropathic pain such as pain with diabetic neuropathy, pain after virus infections or pain after nerve lesions, hyperalgesia, allodynia, idiopathic pain, visceral pain, abdominal pain, and toothache.

In particular, the composition according to Embodiment 1 may be used for the treatment of acute and chronic pain, pain of the locomotor system, arthritic and osteoarthritic pain, cancer and tumor pain, postoperative pain, neuropathic pain, migraine, and inflammatory pain.

The composition according to Embodiment 1 is further suitable for the treatment of gastric diseases (inflammation of the stomach, gastric ulcers), intestinal diseases, particularly chronic inflammation of the small and large intestines (irritable colon syndrome—colon irritabile, colitis ulcerosa, Morbus Crohn), diarrhea, constipation, ileus, post-operative ileus, opioid-induced bowel dysfunction and other gastrointestinal motility disorders; rheumatic diseases such as rheumatoid arthritis, osteoarthritis, arthrosis, spondylosis, lumbago, lupus erythematosus and spondylarthropathy; tumors and cancer; obesity and overweight; hepatic disorders, liver inflammatory disorders, obesity and overweight.

Moreover, the composition in accordance with Embodiment 1 is suitable for the withdrawal of drug addiction, such as to opiates, cocaine or alcohol, for the withdrawal of food, buying, internet, computer, phone and gambling addiction, for the treatment of psychic diseases, psychosis, schizophrenia, stress-related conditions (e.g. depression and anxiety), eating disorders and to reduce food intake in humans.

The present invention also relates to a pharmaceutical formulation comprising the composition according to Embodiment 1.

For the rectal administration of the composition according to Embodiment 1, the pharmaceutical formulation according to Embodiment 1 is preferably in the dosage form of a suppository or an enema.

For the sublingual administration of the composition according to Embodiment 1, the pharmaceutical formulation according to Embodiment 1 is preferably in the dosage form of a sublingual tablet, a sublingual film, or a sublingual spray.

For the buccal administration of the composition according to Embodiment 1, the pharmaceutical formulation according to Embodiment 1 is preferably in the dosage form of a buccal tablet, a buccal patch, a buccal film, a buccal liquid, a buccal semisolid, a buccal spray, or a lollipop.

For the peroral administration of the composition according to Embodiment 1, the pharmaceutical formulation according to Embodiment 1 is preferably in the dosage form of a tablet, a pill, a dragée, a capsule, a softgel capsule.

For the transdermal administration of the composition according to Embodiment 1, the pharmaceutical formulation according to Embodiment 1 is preferably in the form of a transdermal patch (such as reservoir-type and matrix-type patches), microneedles, sonophoresis, electroporation, electro-osmosis, iontophoresis, iontophoresis patch, short-duration shock waves, photomechanical waves.

For the nasal administration of the composition according to Embodiment 1, the pharmaceutical formulation according to Embodiment 1 is preferably in the dosage form of a spray (such as liquid spray, powder spray), squirt system or drops.

In a particularly preferred embodiment, which may be combined with any of the preceding or following embodiments, the aforementioned tablet, pill, dragée, capsule, or softgel capsule has an enteric coating allowing a controlled release of the pharmaceutically active compound (a) by targeting target later segments of the gastrointestinal tract by using an enteric coating known in the art that dissolves at higher pH values.

The pharmaceutical formulation comprising the composition according to Embodiment 1 comprises in a preferred embodiment, which may be combined with any of the preceding or following embodiments, 0.1 to 1,000 mg of component (a). In a more preferred embodiment, it comprises 1 to 500 mg of component (a), in an even more preferred embodiment 1.5 to 300 mg, and in a particularly preferred embodiment, which may be combined with any of the preceding and following embodiments, 2 to 200 mg of component (a).

The pharmaceutical formulation comprising the composition according to Embodiment 1 may be produced in accordance with established procedures known by the person skilled in the art.

Embodiment 1 shall be illustrated further in the following examples.

### EXAMPLE 1-1: PERMEATION STUDIES OF 2-[(4,5α-EPOXY-3-HYDROXY-14β-METHOXY-17-METHYLMORPHINAN-6β-YL)AMINO]ACETIC ACID DIHYDROCHLORIDE HYDRATE ETHANOLATE (“HS731”) IN THE ABSENCE OF A PERMEATION ENHANCER

Ussing type diffusion chambers with a surface area of 0.64 cm² were utilized. Rat small intestine was excised immediately after sacrificing the animal and mounted in the chamber. The donor and acceptor compartments of the chamber were filled with 1.0 ml of the freshly prepared incubation medium containing either 40 mM HEPES buffer (HEPES=2-(4-(2-hydroxyethyl)-1-piperazinyl)-ethane sulfonic acid) adjusted to pH 7.5 or 6.5 or 50 mM sodium acetate buffer adjusted to pH 5.5. Permeation studies were performed in an atmosphere of 95% O₂ and 5% CO₂ at 37° C. and were started 15 minutes after the mounting of the tissue. For the determination of the permeability of HS731 (synthesized in accordance with to the procedure described for compound 70 in EP-A1-1762569) the solution in the donor chamber, apical side, was replaced by 1.0 ml incubation medium containing 2 mg of HS731. Over three hours incubation period 100 μl samples were withdrawn from the acceptor chamber at the basolateral side every 30 minutes, and the volume was replaced by the same medium (without HS731) equilibrated to 37° C. The samples were centrifuged at 7,200 g for 5 minutes and the supernatant was analyzed via HPLC. Cumulative corrections were performed for the previously removed samples to determine the total amount permeated. Results of the permeation studies from apical to basolateral side through rat intestinal mucosa displayed a greater permeation of HS731 at pH 7.5 followed by pH 6.5 and pH 5.5. At pH 7.5 HS731 showed the highest permeation (11%) through rat intestinal mucosa. Therefore the permeation studies in the presence of permeation enhancers were performed at pH 7.5.

### EXAMPLE 1-2: PERMEATION STUDIES WITH HS731 AFTER ADDITION OF A PERMEATION ENHANCER

The permeation of HS731 was performed using 40 mM HEPES buffer adjusted to pH 7.5 as described in Example 1 in the presence of different permeation enhancers (Table 1-2).

The middle chain fatty acid capric acid, the sodium salt of lauric acid and the surfactant Cremophor® EL was added to the donor chamber in a concentration of 1% (m/v) (Table 1-2, entries 1 to 3). Additional results were gathered for the use of the polymer poly(acrylic acid) (PAA₄₅₀) as well as its thiol group bearing conjugate poly(acrylic acid)-cysteine (PAA₄₅₀-Cys) in a concentration of 0.5% (m/v) (Table 1-2, entries 4 and 5). Finally, capric acid was added in a concentration of 0.25% (m/v) (Table 1-2, entry 6).

TABLE 1-2
Exemplary compositions of
HS731 and different permeation enhancers.

| | | | | Concentration |
|---|---|---|---|---|
| | | Active | | Permeation |
| Entry | Composition | Compound | Permeation Enhancer | Enhancer |
| 1 | A | HS731 | Capric Acid | 1% (m/v) |
| | | | [Sigma, Austria] | |
| 2 | B | HS731 | Cremophor ® EL | 1% (m/v) |
| | | | [Fluka, Austria] | |
| 3 | C | HS731 | Sodium laureate | 1% (m/v) |
| | | | [Fluka, Austria] | |
| 4 | D | HS731 | PAA₄₅₀ | 0.5% (m/v) |
| | | | [Sigma, Austria] | |
| 5 | E | HS731 | PAA₄₅₀-Cys | 0.5% (m/v) |
| | | | [ThioMatrix, Austria] | |
| 6 | F | HS731 | Capric acid | 0.25% (m/v) |
| | | | [Sigma, Austria] | |

Enhanced permeation was achieved with all permeation enhancers used (Table 1-3). The results show in general a permeation enhancing effect with an enhancement ratio (ER) range from 1.2 to 2.4. Capric acid and Cremophor® EL in a concentration of 1% (m/v) led to a greater permeation of HS731 compared to the other enhancers (Table 1-3, entries 1 and 2). No significant differences between the permeation of HS731 in addition of 0.5% (m/v) PAA₄₅₀, 0.5% (m/v) PAA₄₅₀-Cys and 0.25% (m/v) capric acid were observed (Table 1-3, entries 3 to 5).

TABLE 1-3
Enhancement ratios (ER) for the permeation of HS731 in the
presence of different permeation enhancers.

| Entry | Composition | Enhancement Ratio (ER) |
|---|---|---|
| 1 | A | 2.4 |
| 2 | B | 1.9 |
| 3 | D | 1.8 |
| 4 | F | 1.7 |
| 5 | E | 1.6 |
| 6 | C | 1.2 |

### EXAMPLE 1-3: FURTHER OPIOID RECEPTOR AGONISTS

The c log P and log D values were calculated with MarvinSketch software [http://www.chemaxon.com/products/marvin/marvinsketch/].

[³⁵S]GTPγS binding was performed as described in: M. Spetea et al., J. Med. Chem. 2011, 54, 980-988. Opioid receptor binding was performed as described in: M. Spetea et al., Eur. J. Pharmacol. 2004, 483, 301-308

TABLE 1-4
Physiochemical properties and opioid receptor activities of agonists

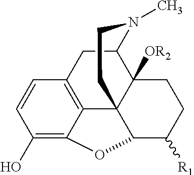

| | | | | | | | | | [³⁵S]GTPγS | |
|---|---|---|---|---|---|---|---|---|---|---|
| | | | | | | | | | binding, MOR^{c} | |
| | R₁ | 6-NH | | | clog D^{a} | Binding Kᵢ (nM)^{b} | | | EC₅₀ | % |
| Compound | (AA) | α/β | R₂ | clog P^{a} | (pH 7.4) | MOR | DOR | KOR | (nM) | stim.^{d} |
| Morphine | OH | | H | | | 6.55 | 217 | 113 | 36 | 106 |
| OMO | O | | H | 0.85 | 0.01 | 0.97 | 80.5 | 61.6 | 4.40 | 98 |
| 14-OMO | O | | Me | 1.53 | 0.56 | 0.10 | 4.80 | 10.2 | 1.17 | 127 |
| HS730 | Gly | α | Me | −1.68 | −2.70 | 0.89 | 15.4 | 43.2 | — | — |
| HS731 | Gly | β | Me | −1.68 | −2.70 | 0.83 | 7.86 | 44.8 | 3.88 | 107 |
| 1 | L-Ala | α | Me | −1.12 | −2.13 | 0.77 | 26.9 | 142 | — | — |
| 2 | L-Ala | β | Me | −1.12 | −2.13 | 1.90 | 7.71 | 63.7 | — | — |
| 3 | L-Phe | α | Me | 0.54 | −0.48 | 0.95 | 3.67 | 28.5 | 0.03 | 116 |
| 4 | L-Phe | β | Me | 0.54 | −0.48 | 2.58 | 1.03 | 151 | — | — |
| 5 | Gly | α | Et | −1.33 | −2.31 | 0.57 | 10.3 | 45.2 | — | — |
| 6 | Gly | β | Et | −1.33 | −2.31 | 0.95 | 5.31 | 102 | — | — |
| 7 | Gly | α | PP | 0.77 | −0.17 | 0.19 | 0.22 | 0.73 | — | — |
| 8 | Gly | β | PP | 0.77 | −0.17 | 0.16 | 0.19 | 0.81 | — | — |
| 9 | L-Tyr | α | Me | 0.24 | −0.78 | 0.83 | 2.18 | 39.5 | — | — |
| 10 | L-Tyr | β | Me | 0.24 | −0.78 | 3.20 | 3.89 | 186 | — | — |
| 11 | L-Phe | α | H | −0.10 | −1.46 | 2.30 | 19.0 | 344 | — | — |
| 12 | L-Phe | β | H | −0.10 | −1.46 | 6.91 | 8.29 | 634 | — | — |
| 13 | L-Ala | α | H | −1.76 | −3.12 | 3.00 | 170 | 710 | — | — |
| 14 | L-Ala | β | H | −1.76 | −3.12 | 11.0 | 78.7 | 542 | — | — |
| 15 | GABA | α | Me | −1.26 | −2.29 | 0.77 | 12.5 | 45.6 | 1.90 | 113 |
| 16 | GABA | β | Me | −1.26 | −2.29 | 1.41 | 6.61 | 147 | — | — |
| 17 | L-Gln | α | Me | −2.28 | −3.29 | 3.24 | 5.13 | 351 | — | — |
| 18 | L-Gln | β | Me | −2.28 | −3.29 | 2.48 | 4.87 | 290 | — | — |
| 19 | L-Glu | α | Me | −1.47 | −4.37 | 1.45 | 9.03 | 87.2 | 1.61 | 105 |
| 20 | L-Glu | β | Me | −1.47 | −4.37 | 11.6 | 7.64 | 1252 | — | — |
| 21 | L-β-Ala | α | Me | −1.44 | −2.48 | 1.30 | 60.0 | 182 | — | — |
| 22 | L-β-Ala | β | Me | −1.44 | −2.48 | 1.04 | 13.9 | 71.4 | — | — |
| 23 | L-Met | α | Me | −0.46 | −1.48 | 0.93 | 4.03 | 109 | — | — |
| 24 | L-Met | β | Me | −0.46 | −1.48 | 3.88 | 2.40 | 468 | — | — |
| 25 | Gly-Gly | β | Me | −3.10 | −4.14 | 4.62 | 7.52 | 203 | — | — |
| 26 | L-Asn | α | Me | −2.56 | −3.56 | 1.17 | 3.37 | 74.0 | 0.64 | 109 |
| 27 | L-Asn | β | Me | −2.56 | −3.56 | 1.26 | 2.25 | 103 | — | — |
| 28 | L-Val | α | Me | −0.23 | −1.24 | 3.16 | 3.91 | 325 | — | — |
| 29 | L-Val | β | Me | −0.23 | −1.24 | 3.04 | 3.52 | 305 | — | — |
| 30 | D-Val | α | Me | −0.23 | −1.24 | 1.70 | 1.93 | 202 | — | — |
| 31 | D-Val | β | Me | −0.23 | −1.24 | 1.02 | 1.68 | 159 | — | — |
| 32 | L-Val-L-Tyr | α | Me | 0.28 | −0.75 | 0.82 | 1.19 | 69.0 | — | — |
| 33 | L-Val-L-Tyr | β | Me | 0.28 | −0.75 | 0.44 | 1.38 | 390 | — | — |
| 34 | L-Thr | α | Me | −1.75 | −2.76 | 1.03 | 4.13 | 120 | — | — |
| 35 | L-Thr | β | Me | −1.75 | −2.76 | 0.79 | 5.16 | 58.6 | — | — |
| 36 | L-Ser | α | Me | −2.16 | −3.18 | 2.21 | 5.32 | 196 | 1.21 | 96 |
| 37 | L-Ser | β | Me | −2.16 | −3.18 | 2.14 | 5.29 | 152 | 3.24 | 101 |
| 38 | L-Lys | α | Me | −1.25 | −4.85 | 0.19 | 1.27 | 12.6 | — | — |
| 39 | L-Lys | β | Me | −1.25 | −4.85 | 0.53 | 3.34 | 33.7 | — | — |
| 40 | L-Leu | α | Me | 0.14 | −0.88 | 0.68 | 2.34 | 141 | — | — |
| 41 | L-Leu | β | Me | 0.14 | −0.88 | 1.32 | 1.01 | 297 | — | — |
| 42 | L-Ile | α | Me | 0.22 | −0.80 | 0.84 | 3.20 | 131 | — | — |
| 43 | L-Ile | β | Me | 0.22 | −0.80 | 1.46 | 1.30 | 163 | — | — |
| 44 | L-Asp | α | Me | −1.76 | −4.65 | 1.36 | 14.6 | 50.2 | — | — |
| 45 | L-Asp | β | Me | −1.76 | −4.65 | 3.42 | 22.6 | 351 | — | — |
| 46 | L-Trp | β | Me | 0.64 | −0.38 | 0.65 | 1.19 | 8.66 | 0.61 | 112 |
| 47 | L-Abu | α | Me | −0.59 | −1.61 | 0.76 | 37.5 | 144 | — | — |
| 48 | L-Abu | β | Me | −0.59 | −1.61 | 1.83 | 1.30 | 201 | — | — |
| 49 | L-Chg | α | Me | 0.64 | −0.37 | 1.23 | 14.3 | 177 | — | — |
| 50 | L-Chg | β | Me | 0.64 | −0.37 | 1.66 | 1.30 | 118 | — | — |
| 51 | D-Phe | α | Me | 0.54 | −0.48 | 0.61 | 3.69 | 76.4 | — | — |
| 52 | D-Phe | β | Me | 0.54 | −0.48 | 1.28 | 1.19 | 139 | — | — |
| 53 | D-Ala | α | Me | −1.12 | −2.13 | 0.69 | 10.4 | 71.5 | — | — |
| 54 | D-Ala | β | Me | −1.12 | −2.13 | 1.48 | 11.3 | 142 | — | — |

^{a}Calculated with MarvinSketch software; ^{b}Determined in in vitro binding assays in rat brain (MOR and DOR) or guinea pig brain (KOR) membranes; ^{c}Stimulation of [³⁵S]GTPγS binding to human MOP expressed in CHO cells; ^{d}Measured against DAMGO; AA: amino acid residue; OMO: oxymorphone; 14-OMO: 14-O-methyloxymorphone; PP: phenylpropyl; —: not tested; morphine, OMO and 14-OMO are reference compounds;

Most of the 6-(amino acid)-morphinans (compounds 1-54, HS730 and HS731) show high binding affinities to the MOR (Kᵢ values in the nanomolar and subnanomolar range), while DOR binding is mostly somewhat lower, and KOR binding in most cases considerably lower. A number of compounds were evaluated for agonist potency and efficacy at the MOR using the [³⁵S]GTPγS functional assay in CHO cells expressing human MOR receptors. These compounds show high agonist potency and efficacy. According to the calculated log D value, the listed compounds (1-54) have a lipophilicity similar to HS731 and thus addition of a permeation enhancer produces a superior bioavailability after oral, rectal, transdermal and/or nasal administration. In the prior art, satisfactory oral bioavailability has been attributed only to compounds with a c log D value of above 1. (Tetko I. V. and Poda G I, J. Med. Chem. 2004, 47, 5601-5604) Accordingly, the compounds according to embodiment 1 are to be considered as compounds of low bioavailability, which, together with their high hydrophilicity, severely impairs their therapeutic use, as the prior art does not disclose a concept for reaching satisfactory bioavailability for this type of compounds.

TABLE 1-5
Ionization constants and octanol-water partition (logP)
and distribution (logD) coefficients of selected opioid morphinans

| | | | logD |
|---|---|---|---|
| Compound | pKₐ | logP | (pH = 7.4) |
| Morphine^{a)} | 8.15 | 0.88 | 0.06 |
| Oxymorphone^{a)} | 8.33 | 0.67 | −0.32 |
| 14-O-Methyloxymorphone^{a)} | 8.18 | 0.60 | −0.25 |
| 14-Methoxymetopon^{a)} | 8.36 | 1.12 | 0.11 |
| HS731 | 2.4 | −0.78 | −2.06 |
| 35 | ND | −1.38 | ND |
| 15 | ND | −0.36 | ND |
| 38 | ND | −0.66 | ND |
| 45 | ND | −2.20 | ND |

ND = not determined
^{a)}Data of these compounds have been published: Riba P. et al. Brain Res. Bull. 2010, 81, 178-184

Lipophilicity of opioid morphinans was evaluated at 25° C. by determination of partition coefficient (log P) and distribution coefficient (log D) in an immiscible (biphasic) octanol/water medium using the PCA200/Cheqsol instrument (Sirius Analytical Instruments, Sussex, UK). All titrations were performed in 0.15M KCl solution under argon gas. The pKa was determined by the shape of the titration curve and partition coefficients were determined by the shift in titration curve of pKₐ in the presence of octanol. The analysis of pH-metric data was performed by RefinementPro software (version 2.0, Sirius, Sussex, UK).

The experimentally obtained log P values of 6-amino acid substituted morphinans with a zwitterionic amino acid moiety (such as HS731), which makes them extremely hydrophilic, are considerably lower than for morphine, oxymorphone, 14-O-methyloxymorphone and 14-methoxymetopon. For HS731 an experimentally pKa value of 2.4 and a log D value of −2.06 was found.

Morphine has an oral bioavailability of about 40% and is used typically without penetration entrances. Calculated values of hydromorphone, which is structurally closely related to morphine, oxymorphone, 14-O-methyloxymorphone and 14-methoxymetopon, are: c log P 1.62 and pkₐ 8.59 (Marvin Sketch). These calculated values of hydromorphone are somewhat higher compared to the experimentally obtained values of the other structurally related compounds, morphine, oxymorphone, 14-O-methyloxymorphone and 14-methoxymetopon.

In any case, these values do show that the compounds to be used according to the invention differ vastly with respect to relevant properties from well known compounds used as reference herein.

### EXAMPLE 1-4: OPIOID RECEPTOR ANTAGONISTS

The c log P and log D values were calculated with MarvinSketch software [http://www.chemaxon.com/products/marvin/marvinsketch/].

[³⁵S]GTPγS binding was performed as described: M. Spetea et al. Life Sci. 2001, 69, 1775-1782. Opioid receptor binding was performed as described in: M. Spetea et al., Eur. J. Pharmacol. 2004, 483, 301-308

TABLE 1-6
Physiochemical properties and opioid receptor activities of antagonists

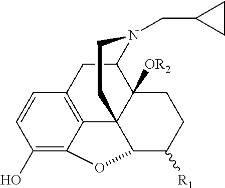

| | | | | | | | | | [³⁵S]GTPγS | |
|---|---|---|---|---|---|---|---|---|---|---|
| | | | | | | | | | binding, MOR^{c} | |
| | R₁ | 6-NH | | | clog D^{a} | Binding Kᵢ (nM)^{b} | | | EC₅₀ | Kₑ |
| Compound | (AA) | α/β | R₂ | clog P^{a} | (pH 7.4) | MOR | DOR | KOR | (nM) | (nM)^{d} |
| NTX | O | — | H | 1.67 | 0.13 | 0.54 | 14.8 | 0.81 | >10,000 | 0.59 |
| 14-OMe-NTX | O | — | Me | 2.31 | 0.68 | 0.46 | 7.88 | 1.17 | >10,000 | 0.49 |
| 14-OEt-NTX | O | — | Et | 2.67 | 0.98 | 0.35 | 4.13 | 0.93 | >10,000 | 0.31 |
| 59 | L-Phe | α | H | 0.68 | −1.36 | 1.41 | 3.53 | 2.65 | >10,000 | 0.25 |
| 60 | L-Phe | β | H | 0.68 | −1.36 | 1.83 | 2.79 | 8.99 | >10,000 | 0.24 |
| 61 | Gly | α | Me | −0.90 | −1.57 | 0.68 | 5.15 | 4.65 | >10,000 | 0.63 |
| 62 | Gly | β | Me | −0.90 | −1.57 | 1.31 | 4.76 | 9.31 | >10,000 | 0.19 |
| 63 | L-β-Ala | α | Me | −0.66 | −1.34 | 1.35 | 6.25 | 7.81 | >10,000 | 0.27 |
| 64 | L-β-Ala | β | Me | −0.66 | −1.34 | 1.51 | 8.12 | 11.7 | >10,000 | 0.96 |
| 65 | GABA | α | Me | −0.48 | −1.16 | 1.21 | 10.7 | 5.31 | >10,000 | 0.94 |
| 66 | GABA | β | Me | −0.48 | −1.16 | 3.36 | 10.2 | 16.8 | >10,000 | 1.06 |
| 67 | Gly | α | H | −1.55 | −3.58 | 0.88 | 13.1 | 2.81 | — | — |
| 68 | L-Val | α | Me | 0.55 | −0.11 | 0.44 | 0.53 | 3.34 | — | — |
| 69 | L-Val | β | Me | 0.55 | −0.11 | 0.66 | 1.35 | 3.00 | — | — |
| 70 | D-Val | α | Me | 0.55 | −0.11 | 0.62 | 0.71 | 3.54 | — | — |
| 71 | D-Val | β | Me | 0.55 | −0.11 | 1.52 | 0.35 | 23.0 | — | — |
| 72 | L-Ala | β | Me | −0.33 | −1.00 | 2.13 | 5.80 | 11.5 | — | — |
| 73 | L-Asp | β | Me | −0.98 | −3.91 | 2.24 | 0.64 | 11.7 | — | — |
| 74 | L-Glu | α | Me | −0.69 | −3.63 | 1.93 | 3.61 | 6.66 | — | — |
| 75 | L-Glu | β | Me | −0.69 | −3.63 | 3.35 | 2.77 | 12.9 | — | — |
| 79 | L-Abu | β | Me | 0.19 | −0.48 | 3.39 | 6.20 | 19.1 | — | — |

^{a}Calculated with MarvinSketch software; ^{b}Determined in in vitro binding assays in rat brain (MOR and DOR) or guinea pig brain (KOR) membranes; ^{c}Stimulation of [³⁵S]GTPγS binding to human MOP expressed in CHO cells; ^{d}Measured against DAMGO; AA: amino acid residue; NTX: Naltrexone; 14-OMe-NTX: 14-O-Methylnaltrexone; 14-OEt-NTX: 14-O-Ethylnaltrexone; —: not tested; NTX, 14-OMe-NTX und 14-OEt-NTX are reference compounds.

All 6-(amino acid)-morphinans (compounds 55-79) show high binding affinities to the MOR (Kᵢ values in the nanomolar and subnanomolar range), while DOR binding is mostly somewhat lower and KOR binding lower. In the functional assay, [³⁵S]GTPγS binding at MOR, the 6-(amino acid)-morphinans (compounds 59-66) exhibit antagonist Kₑ values in the subnanomolar or low nanomolar range, thus proving high antagonism mediated via the MORs.

### Embodiment 2

Optimizing the bioavailability of a candidate molecule is a key objective in drug discovery programs. Clearly, compounds exhibiting low oral bioavailability are likely to require high doses to achieve the desired effects, since systematic exposure to the active compound will be limited. Optimal physicochemical properties to allow high transcellular absorption are well established and include a limit on molecular size, hydrogen bonding potential and adequate lipophilicity. Hydrogen bonding groups of an active compound can be masked by addition of another moiety, most commonly an ester in order to increase lipophilicity. Such bioreversible derivatives have considerable higher bioavailability and usually undergo enzymatic cleavage by enzymes to regenerate the parent drug after absorption [K. Beaumont, R. Webster, I. Gardner, K. Dack. Curr. Drug Metab. 2003, 4, 461-485].

The above defined object is thus also solved by a compound of Formula (II) for use in an orally, rectally, transdermally or nasally administered medicament,

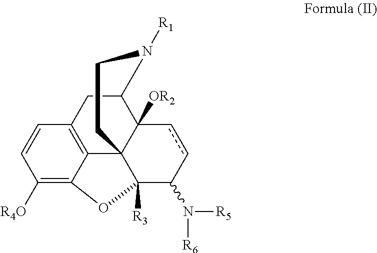

wherein the substituents R₁, R₂, R₃, R₄, R₅ and R₆ have the following meaning:

R₁ is selected from hydrogen; C1-C30, preferably C1-C12, more preferably C₁-C₆-alkyl; C2-C30, preferably C2-C12, more preferably C₂-C₆-alkenyl; C2-C30, preferably C2-C12, more preferably C₂-C₆-alkynyl; C1-C30, preferably C1-C12, more preferably C₁-C₆-monohydroxyalkyl; C2-C30, preferably C2-C12, more preferably C₂-C₆-dihydroxyalkyl; C3-C30, preferably C3-C12, more preferably C₃-C₆-trihydroxyalkyl; C4-C30, preferably C₄-C₁₆-cycloalkylalkyl, wherein cycloalkyl preferably is C₃-C₁₀-cycloalkyl and alkyl preferably is C₁-C₆-alkyl; C5-C30, preferably C₅-C₁₆-cycloalkylalkenyl, wherein cycloalkyl preferably is C₃-C₁₀-cycloalkyl and alkenyl preferably is C₂-C₆-alkenyl; C5-C30, preferably C₅-C₁₆-cycloalkylalkynyl, wherein cycloalkyl preferably is C₃-C₁₀-cycloalkyl and alkynyl preferably is C₂-C₆-alkynyl; C7-C30, preferably C₇-C₁₆-arylalkyl, wherein aryl preferably is C₆-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; C8-C30, preferably C₈-C₁₆-arylalkynyl, wherein aryl preferably is C₆-C₁₀-aryl and alkynyl preferably is C₂-C₆-alkynyl;

R₂ is selected from hydrogen; C1-C30, preferably C1-C12, more preferably C₁-C₆-alkyl; C1-C30, preferably C1-C12, more preferably C₁-C₆-monohydroxyalkyl; C2-C30, preferably C2-C12, more preferably C₂-C₆-dihydroxyalkyl; C3-C30, preferably C3-C12, more preferably C₃-C₆-trihydroxyalkyl; C2-C30, preferably C2-C12, more preferably C₂-C₆-alkenyl; C2-C30, preferably C2-C12, more preferably C₂-C₆-alkynyl; C4-C30, preferably C₄-C₁₆-cycloalkylalkyl, wherein cycloalkyl preferably is C₃-C₁₀-cycloalkyl and alkyl preferably is C₁-C₆-alkyl; C5-C30, preferably C₅-C₁₆-cycloalkylalkenyl, wherein cycloalkyl preferably is C₃-C₁₀-cycloalkyl and alkenyl preferably is C₂-C₆-alkenyl; C5-C30, preferably C₅-C₁₆-cycloalkylalkynyl, wherein cycloalkyl preferably is C₃-C₁₀-cycloalkyl and alkynyl preferably is C₂-C₆-alkynyl; C7-C30, preferably C₇-C₁₆-arylalkyl, wherein aryl preferably is C₆-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; C8-C30, preferably C₈-C₁₆-arylalkynyl, wherein aryl preferably is C₆-C₁₀-aryl and alkynyl preferably is C₂-C₆-alkynyl; C2-C30, preferably C2-C12, more preferably C₂-C₆-alkanoyl; C3-C30, preferably C3-C12, more preferably C₃-C₆-alkenoyl; C3-C30, preferably C3-C12, more preferably C₃-C₆-alkinoyl; C7-C30, preferably C₇-C₁₆-arylalkanoyl, wherein aryl preferably is C₆-C₁₀-aryl and alkanoyl preferably is C₁-C₆-alkanoyl; C9-C30, preferably C₉-C₁₆-arylalkenoyl, wherein aryl preferably is C₆-C₁₀-aryl and alkenoyl preferably is C₃-C₆-alkenoyl; C9-C30, preferably C₉-C₁₆-arylalkinoyl, wherein aryl preferably is C₆-C₁₀-aryl and alkinoyl preferably is C₃-C₆-alkinoyl;

R₃ is selected from hydrogen; C1-C30, preferably C1-C12, more preferably C₁-C₆-alkyl; C2-C30, preferably C2-C12, more preferably C₂-C₆-alkenyl; C7-C30, preferably C₇-C₁₆-arylalkyl, wherein aryl preferably is C₆-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; alkoxyalkyl, wherein alkoxy is C₁-C₆-alkoxy and alkyl is C₁-C₆-alkyl; CO₂(C₁-C₆-alkyl); CO₂H; CH₂OH;

R₄ is selected from hydrogen; C1-C30, preferably C1-C12, more preferably C₁-C₆-alkyl; C2-C30, preferably C2-C12, more preferably C₂-C₆-alkenyl; C2-C30, preferably C2-C12, more preferably C₂-C₆-alkynyl; C4-C30, preferably C₄-C₁₆-cycloalkylalkyl, wherein cycloalkyl preferably is C₃-C₁₀-cycloalkyl and alkyl preferably is C₁-C₆-alkyl; C5-C30, preferably C₅-C₁₆-cycloalkylalkenyl, wherein cycloalkyl preferably is C₃-C₁₀-cycloalkyl and alkenyl preferably is C₂-C₆-alkenyl; C5-C30, preferably C₅-C₁₆-cycloalkylalkynyl, wherein cycloalkyl preferably is C₃-C₁₀-cycloalkyl and alkynyl preferably is C₂-C₆-alkynyl; C2-C30, preferably C2-C12, more preferably C₂-C₆-alkenyl; C2-C12, preferably C₂-C₆-alkynyl; C7-C30, preferably C₇-C₁₆-arylalkyl, wherein aryl preferably is C₆-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; C8-C30, preferably C₈-C₁₆-arylalkynyl, wherein aryl preferably is C₆-C₁₀-aryl and alkynyl preferably is C₂-C₆-alkynyl; C2-C30, preferably C2-C12, more preferably C₂-C₆-alkanoyl; C3-C30, preferably C3-C12, more preferably C₃-C₆-alkenoyl; C3-C30, preferably C3-C12, more preferably C₃-C₆-alkinoyl; C7-C30, preferably C₇-C₁₆-arylalkanoyl, wherein aryl preferably is C₆-C₁₀-aryl and alkanoyl preferably is C₁-C₆-alkanoyl; C9-C30, preferably C₉-C₁₆-arylalkenoyl, wherein aryl preferably is C₆-C₁₀-aryl and alkenoyl preferably is C₃-C₆-alkenoyl; C9-C30, preferably C₉-C₁₆-arylalkinoyl, wherein aryl preferably is C₆-C₁₀-aryl and alkinoyl preferably is C₃-C₆-alkinoyl; iminomethyl, formamidinyl, C1-C30, preferably C1-C12, more preferably C₁-C₆—N-alkyl- and N,N′-dialkylformamidinyl; C2-C30, preferably C2-C12, more preferably C₂-C₆—N-alkenyl- and N,N′-dialkenylformamidinyl; C2-C30, preferably C2-C12, more preferably C₂-C₆—N-alkynyl- and N,N′-dialkynylformamidinyl; C4-C30, preferably C₄-C₁₆—N-cycloalkylalkyl- and N,N′-dicycloalkylalkylformamidinyl, wherein cycloalkyl preferably is C₃-C₁₀-cycloalkyl and alkyl preferably is C₁-C₆-alkyl; C5-C30, preferably C₅-C₁₆—N-cylcoalkylalkenyl- and N,N′-dicycloalkylalkenylformamidinyl, wherein cycloalkyl preferably is C₃-C₁₀-cycloalkyl and alkenyl preferably is C₂-C₆-alkenyl; C5-C30, preferably C₅-C₁₆—N-cycloalkylalkynyl- and N,N′-dicycloalkylalkynylformamidinyl, wherein cycloalkyl preferably is C₃-C₁₀-cycloalkyl and alkynyl preferably is C₂-C₆-alkynyl; C7-C30, preferably C₇-C₁₆—N-arylalkyl- and N,N′-diarylalkylformamidinyl, wherein aryl preferably is C₆-C₁₀-aryl and alkyl preferably is C₁-C₆-alkyl;

R₅ is selected from hydrogen, C1-C30, preferably C1-C12, more preferably C₁-C₆-alkyl; C2-C30, preferably C2-C12, more preferably C₂-C₆-alkenyl; C2-C30, preferably C2-C12, more preferably C₂-C₆-alkynyl; C4-C30, preferably C₄-C₁₆-cycloalkylalkyl, wherein cycloalkyl preferably is C₃-C₁₀-cycloalkyl and alkyl preferably is C₁-C₆-alkyl; C5-C30, preferably C₅-C₁₆-cycloalkylalkenyl, wherein cycloalkyl preferably is C₃-C₁₀-cycloalkyl and alkenyl preferably is C₂-C₆-alkenyl; C5-C30, preferably C₅-C₁₆-cycloalkylalkynyl, wherein cycloalkyl preferably is C₃-C₁₀-cycloalkyl and alkynyl preferably is C₂-C₆-alkynyl; C7-C30, preferably C₇-C₁₆-arylalkyl, wherein aryl preferably is C₅-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; C8-C30, preferably C₈-C₁₆-arylalkynyl, wherein aryl preferably is C₆-C₁₀-aryl and alkynyl preferably is C₂-C₆-alkynyl;

CH(A)CO₂B, wherein A is selected from hydrogen; hydroxyl; C1-C30, preferably C1-C12, more preferably C₁-C₆-alkyl; C2-C30, preferably C2-C12, more preferably C₂-C₆-alkenyl; C2-C30, preferably C2-C12, more preferably C₂-C₆-alkynyl; C4-C30, preferably C₄-C₁₆-cycloalkylalkyl, wherein cycloalkyl preferably is C₃-C₁₀-cycloalkyl and alkyl preferably is C₁-C₆-alkyl; C5-C30, preferably C₅-C₁₆-cycloalkylalkenyl, wherein cycloalkyl preferably is C₃-C₁₀-cycloalkyl and alkenyl preferably is C₂-C₆-alkenyl; C5-C30, preferably C₅-C₁₆-cycloalkylalkynyl, wherein cycloalkyl preferably is C₃-C₁₀-cycloalkyl and alkynyl preferably is C₂-C₆-alkynyl; C7-C30, preferably C₇-C₁₆-arylalkyl, wherein aryl preferably is C₆-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; C8-C30, preferably C₈-C₁₆-arylalkynyl, wherein aryl preferably is C₆-C₁₀-aryl and alkynyl preferably is C₂-C₆-alkynyl; amino; C1-C30, preferably C1-C12, more preferably C₁-C₆-alkylamino; guanidino; C1-C30, preferably C1-C12, more preferably C₁-C₆-alkyl-CO₂B; C₁-C₆-monohydroxyalkyl; C₂-C₆-dihydroxyalkyl; C₃-C₆-trihydroxyalkyl; C₁-C₆-monoaminoalkyl; C₂-C₆-diaminoalkyl; C₃-C₆-triaminoalkyl; C₁-C₆-alkylguanidino; C₁-C₆-alkylcarboxamide; C₁-C₆-alkylhydroxycarbonyl; C₁-C₆-sulfhydrylalkyl; C₂-C₁₂-alkylthioalkyl, wherein alkylthio is preferably C₁-C₆ and alkyl is preferably C₁-C₆; and wherein B is selected from hydrogen; C1-C30, preferably C1-C12, more preferably C₁-C₆-alkyl; C2-C30, preferably C2-C12, more preferably C₂-C₆-alkenyl; C2-C30, preferably C2-C12, more preferably C₂-C₆-alkynyl; C4-C30, preferably C₄-C₁₆-cycloalkylalkyl, wherein cycloalkyl preferably is C₃-C₁₀-cycloalkyl and alkyl preferably is C₁-C₆-alkyl; C5-C30, preferably C₅-C₁₆-cycloalkylalkenyl, wherein cycloalkyl preferably is C₃-C₁₀-cycloalkyl and alkenyl preferably is C₂-C₆-alkenyl; C5-C30, preferably C₅-C₁₆-cycloalkylalkynyl, wherein cycloalkyl preferably is C₃-C₁₀-cycloalkyl and alkynyl preferably is C₂-C₆-alkynyl; C7-C30, preferably C₇-C₁₆-arylalkyl, wherein aryl preferably is C₅-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; C8-C30, preferably C₈-C₁₆-arylalkynyl, wherein aryl preferably is C₆-C₁₀-aryl and alkynyl preferably is C₂-C₆-alkynyl; phenyl; substituted phenyl; (C₁-C₆-alkyl)CONH₂; (C₁-C₆-alkyl)CONH(C₁-C₆-alkyl); (C₁-C₆-alkyl)CONH(C₁-C₆-alkyl-CONH₂); (C₁-C₆-alkyl)CONHC(C₁-C₆-alkylOH)₃; (C₁-C₆-alkyl)CON(C₁-C₆-alkyl)(heterocyclic ring); (C₁-C₆-alkyl)CONH-heterocyclic ring; (C₁-C₆-alkyl)CON(C₁-C₆-alkyl-OH)₂; (C₁-C₆-alkyl)CON(C₁-C₆-alkyl-OH—C₁-C₆-alkyl)₂; (C₁-C₆-alkyl)CON(C₁-C₆-alkyl)(C₁-C₆-alkyl-CONH₂); (C₁-C₆-alkyl)CON(C₁-C₆-alkyl-OH—C₁-C₆-alkyl)₂; (C₁-C₆-alkyl)CON(C₁-C₆-alkyl)₂; (C₁-C₆-alkyl)CON(C₁-C₆-alkyl)₂; (C₁-C₆-alkyl)CONHCO(C₁-C₆-alkyl); (C₁-C₆-alkyl)CON(C₁-C₆-alkyl)N(C₁-C₆-alkyl)₂; (C₁-C₆-alkyl)CON(C₁-C₆-alkyl)CO(C₁-C₆-alkyl); (C₁-C₆-alkyl)CON(C₁-C₆-alkyl)N(C₁-C₆-alkyl)₂; (C₁-C₆-alkyl)CO(C₁-C₆-alkyl); (C₁-C₆-alkyl)N(C₁-C₆-alkyl)₂; (C₁-C₆-alkyl)N(C₁-C₆-alkyl)CO(C₁-C₆-alkyl); (C₁-C₆-alkyl)OCO(C₁-C₆-alkyl); (C₁-C₆-alkyl)OO(C₁-C₆-alkyl); (C₁-C₆-alkyl)COO(C₁-C₆-alkyl); (C₁-C₆-alkyl)CO₂H; (C₁-C₆-alkyl)OCOO(C₁-C₆-alkyl); (C₁-C₆-alkyl)S(C₁-C₆-alkyl); (C₁-C₆-alkyl)SO(C₁-C₆-alkyl); (C₁-C₆-alkyl)SO₂(C₁-C₆-alkyl); phthalidyl, (5-methyl-2-oxo-1,3-dioxol-4-yl)methyl;-

CH(A)SO₃B, wherein A and B are defined as above;

CH(A)COB1, wherein A is defined as above, and B1 is NH₂; NH—C1-C12, more preferably NH—C₁-C₆-alkyl; N—(C1-C12)₂, more preferably N—(C₁-C₆)₂-alkyl;

R₆ is selected from CH(A)CO₂B, wherein A is defined as above, and wherein B is selected from C1-C30, preferably C1-C12, more preferably C₁-C₆-alkyl; C2-C30, preferably C2-C12, more preferably C₂-C₆-alkenyl; C2-C30, preferably C2-C12, more preferably C₂-C₆-alkynyl; C4-C30, preferably C₄-C₁₆-cycloalkylalkyl, wherein cycloalkyl preferably is C₃-C₁₀-cycloalkyl and alkyl preferably is C₁-C₆-alkyl; C5-C30, preferably C₅-C₁₆-cycloalkylalkenyl, wherein cycloalkyl preferably is C₃-C₁₀-cycloalkyl and alkenyl preferably is C₂-C₆-alkenyl; C5-C30, preferably C₅-C₁₆-cycloalkylalkynyl, wherein cycloalkyl preferably is C₃-C₁₀-cycloalkyl and alkynyl preferably is C₂-C₆-alkynyl; C7-C30, preferably C₇-C₁₆-arylalkyl, wherein aryl preferably is C₅-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; C8-C30, preferably C₈-C₁₆-arylalkynyl, wherein aryl preferably is C₆-C₁₀-aryl and alkynyl preferably is C₂-C₆-alkynyl; phenyl; substituted phenyl; (C₁-C₆-alkyl)CONH₂; (C₁-C₆-alkyl)CONH(C₁-C₆-alkyl); (C₁-C₆-alkyl)CONH(C₁-C₆-alkyl-CONH₂); (C₁-C₆-alkyl)CONHC(C₁-C₆-alkylOH)₃; (C₁-C₆-alkyl)CON(C₁-C₆-alkyl)(heterocyclic ring); (C₁-C₆-alkyl)CONH-heterocyclic ring; (C₁-C₆-alkyl)CON(C₁-C₆-alkyl-OH)₂; (C₁-C₆-alkyl)CON(C₁-C₆-alkyl-OH—C₁-C₆-alkyl)₂; (C₁-C₆-alkyl)CON(C₁-C₆-alkyl)(C₁-C₆-alkyl-CONH₂); (C₁-C₆-alkyl)CON(C₁-C₆-alkyl-OH—C₁-C₆-alkyl)₂; (C₁-C₆-alkyl)CON(C₁-C₆-alkyl)₂; (C₁-C₆-alkyl)CON(C₁-C₆-alkyl)₂; (C₁-C₆-alkyl)CONHCO(C₁-C₆-alkyl); (C₁-C₆-alkyl)CON(C₁-C₆-alkyl)N(C₁-C₆-alkyl)₂; (C₁-C₆-alkyl)CON(C₁-C₆-alkyl)CO(C₁-C₆-alkyl); (C₁-C₆-alkyl)CON(C₁-C₆-alkyl)N(C₁-C₆-alkyl)₂; (C₁-C₆-alkyl)N(C₁-C₆-alkyl)₂; (C₁-C₆-alkyl)N(C₁-C₆-alkyl)CO(C₁-C₆-alkyl); (C₁-C₆-alkyl)OCO(C₁-C₆-alkyl); (C₁-C₆-alkyl)OO(C₁-C₆-alkyl); (C₁-C₆-alkyl)COO(C₁-C₆-alkyl); (C₁-C₆-alkyl)CO₂H; (C₁-C₆-alkyl)OCOO(C₁-C₆-alkyl); (C₁-C₆-alkyl)S(C₁-C₆-alkyl); (C₁-C₆-alkyl)SO(C₁-C₆-alkyl); (C₁-C₆-alkyl)SO₂(C₁-C₆-alkyl); phthalidyl, (5-methyl-2-oxo-1,3-dioxol-4-yl)methyl;

CH(A)SO₃B, wherein A and B are defined as above; and

CH(A)COB1, wherein A is defined as above, and B1 is NH₂; NH—C1-C12, more preferably NH—C₁-C₆-alkyl; N—(C1-C12)₂, more preferably N—(C₁-C₆)₂-alkyl.

Said compounds of Formula (II) may also be designated as “bioreversible esters” of compounds of Formula (I).

Bioreversible alkyl esters of 6-(amino acid)-morphinans may be synthesized according to established procedures in the art which may be derived from the experimental part of EP-A1-1762569. Diesters, acyloxyalkyl esters, aryl esters, glycolamide esters, glycolic esters, glycolate esters, aminoethyl esters may be prepared according to established procedures in the art [H. Bundgaard. In: Design of Prodrugs 1985; H. Bundgaard, N. M. Nielsen. Int. J. Pharmaceutics 1988, 43, 101-110; N. M. Nielsen, H. Bundgaard. J. Pharm. Sciences 1988, 77, 285-298; N. M. Nielsen, H. Bundgaard. J. Med. Chem. 1989, 32, 727-734].

The nitrogen of Formula (II), to which R₁ is attached, may also be substituted by two substituents R₁, which may be the same or different and which are defined as above, and wherein the second, quarternised substituent R₁ may additionally be hydroxyl, oxyl (N-oxide) and alkoxyl.

The term “bioavailability” as used herein has the same meaning as defined for Embodiment 1.

The term “absorption” as used herein has the same meaning as defined for Embodiment 1.

The terms “oral administration”, “rectal administration”, “transdermal administration” and “nasal administration” as used herein have the same meaning as defined for Embodiment 1.

It was surprisingly found herein that compounds according to Formula (II) have an increased bioavailability which allows a reduced administration frequency and/or a reduced dosage of the drug upon administration which significantly contributes to patient compliance and patient satisfaction.

In a preferred embodiment, which may be combined with any of the embodiments described herein, the compound of Formula (II) according to Embodiment 2 is used in an orally, rectally or nasally administered medicament. In a more preferred embodiment, the compound of Formula (II) according to Embodiment 2 is used in an orally or rectally administered medicament.

The dotted line between carbon atoms 7 and 8 of Formula (II) designates that these carbon atoms may be unsaturated (olefinic C—C double bond between C7 and C8) or saturated (C—C single bond between C7 and C8).

Some compounds according to Formula (II) may exist in different stereochemical configurations and/or may show more than one crystalline structure, in particular the compounds possessing one or more chiral carbon atom. Embodiment 2 comprises all those specific embodiments, such as diastereomers, enantiomers, in any given or desired mixture or in isolated form. Furthermore, compounds according to Formula (II) also comprise PEGylated derivatives, poly-L-glutamic acid (PGA) conjugates, N-(2-hydroxypropyl)methacrylamide (HMPA) copolymers, and other polymer conjugates or derivatives thereof as well as polymorphic forms.

The terms “alkyl”, “alkenyl” and “alkynyl” as used herein include both branched and unbranched alkyl, alkenyl and alkynyl groups as well as mono-, di- and trihydroxy-substituted branched and unbranched alkyl, alkenyl and alkynyl groups. These groups furthermore may be substituted once, twice or three times with substituents selected independently from hydroxy, halogen, nitro, cyano, thiocyanato, trifluoromethyl, C₁-C₃-alkyl, C₁-C₃-alkoxy, CO₂H, CONH₂, CO₂(C₁-C₃-alkyl), CONH(C₁-C₃-alkyl), CON(C₁-C₃-alkyl)₂, CO(C₁-C₃-alkyl); amino; (C₁-C₃-monoalkyl)amino, (C₁-C₃-dialkyl)amino, C₅-C₆-cycloalkylamino; (C₁-C₃-alkanoyl)amido, SH, SO₃H, SO₃(C₁-C₃-alkyl), SO₂(C₁-C₃-alkyl), SO(C₁-C₃-alkyl), C₁-C₃-alkylthio or C₁-C₃-alkanoylthio. Further suitable substituents are cyclic groups, including carbocycles and heterocycles which may be saturated, unsaturated or aromatic. Preferred examples comprise from 3 to 8 ring atoms, selected from C, N, O, and S. Aryl can be unsubstituted or mono-, di- or tri-substituted, wherein the substituents can be chosen independently from hydroxy, halogen, nitro, cyano, thiocyanato, trifluoromethyl, C₁-C₃-alkyl, C₁-C₃-alkoxy, CO₂H, CONH₂, CO₂(C₁-C₃-alkyl), CONH(C₁-C₃-alkyl), CON(C₁-C₃-alkyl)₂, CO(C₁-C₃-alkyl); amino; (C₁-C₃-monoalkyl)amino, (C₁-C₃-dialkyl)amino, C₅-C₆-cycloalkylamino; (C₁-C₃-alkanoyl)amido, SH, SO₃H, SO₃(C₁-C₃-alkyl), SO₂(C₁-C₃-alkyl), SO(C₁-C₃-alkyl), C₁-C₃-alkylthio or C₁-C₃-alkanoylthio. Further suitable substituents are cyclic groups, including carbocycles and heterocycles which may be saturated unsaturated or aromatic. Preferred examples comprise from 3 to 8 ring atoms, selected from C, N, O, and S.

The term “aryl” as used herein defines aromatic rings comprising preferably from 5 to 14 ring atoms and the term aryl comprises furthermore carboxylic aryl groups as well as heterocyclic aryl groups, comprising preferably from 1 to 3 heteroatoms, selected from N, O and S. The aryl groups as defined herein may furthermore be fused ring systems such as naphthyl or anthracenyl or the corresponding heterocyclic groups comprising from 1 to 3 heteroatoms selected from N, O, and S.

The above definitions for “alkyl”, “alkenyl”, “alkynyl” and “aryl” are valid for all embodiments of Embodiment 2.

In a preferred embodiment of the compound according to Embodiment 2, which may be combined with any of the preceding and following embodiments, the substituents R₁, R₂, R₃, R₄, R₅ and R₆ of the compound of Formula (II) have the following meaning:

R₁ is selected from hydrogen; C₁-C₁₂-alkyl; C₂-C₁₂-alkenyl; C₂-C₁₂-alkynyl; C₁-C₁₂-monohydroxyalkyl; C₂-C₁₂-dihydroxyalkyl; C₃-C₁₂-trihydroxyalkyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

R₂ is selected from hydrogen; C₁-C₁₂-alkyl; C₁-C₁₂-monohydroxyalkyl; C₂-C₁₂-dihydroxyalkyl; C₃-C₁₂-trihydroxyalkyl; C₂-C₁₂-alkenyl; C₂-C₁₂-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

R₃ is selected from hydrogen; C₁-C₁₂-alkyl; C₂-C₁₂-alkenyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; alkoxyalkyl, wherein alkoxy is C₁-C₆-alkoxy and alkyl is C₁-C₆-alkyl;

R₄ is selected from hydrogen; C₁-C₁₂-alkyl; C₂-C₁₂-alkenyl; C₂-C₁₂-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl; C₂-C₁₂-alkanoyl; C₃-C₁₂-alkenoyl; C₃-C₁₂-alkinoyl; C₇-C₁₆-arylalkanoyl, wherein aryl preferably is C₆-C₁₀-aryl and alkanoyl preferably is C₁-C₆-alkanoyl; C₉-C₁₆-arylalkenoyl, wherein aryl preferably is C₆-C₁₀-aryl and alkenoyl preferably is C₃-C₆-alkenoyl; C₉-C₁₆-arylalkinoyl, wherein aryl preferably is C₆-C₁₀-aryl and alkinoyl preferably is C₃-C₆-alkinoyl;

R₅ is selected from hydrogen, C₁-C₁₂-alkyl; C₂-C₁₂-alkenyl; C₂-C₁₂-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

CH(A)CO₂B, wherein A is selected from hydrogen; hydroxyl; C₁-C₁₂-alkyl; C₂-C₁₂-alkenyl; C₂-C₁₂-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl; amino; C₁-C₆-alkylamino; guanidino; C₁-C₆-alkyl-CO₂B; C₁-C₆-monohydroxyalkyl; C₂-C₆-dihydroxyalkyl; C₃-C₆-trihydroxyalkyl; C₁-C₆-monoaminoalkyl; C₂-C₆-diaminoalkyl; C₃-C₆-triaminoalkyl; C₁-C₆-alkylguanidino; C₁-C₆-alkylcarboxamide; C₁-C₆-alkylhydroxycarbonyl; C₁-C₆-sulfhydrylalkyl; C₂-C₁₂-alkylthioalkyl, wherein alkylthio is preferably C₁-C₆ and alkyl is preferably C₁-C₆; and wherein B is selected from hydrogen; C₁-C₁₂-alkyl; C₂-C₁₂-alkenyl; C₂-C₁₂-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl; phenyl; substituted phenyl; (C₁-C₆-alkyl)CONH₂; (C₁-C₆-alkyl)CONH(C₁-C₆-alkyl); (C₁-C₆-alkyl)CONH(C₁-C₆-alkyl-CONH₂); (C₁-C₆-alkyl)CONHC(C₁-C₆-alkylOH)₃; (C₁-C₆-alkyl)CON(C₁-C₆-alkyl)(heterocyclic ring); (C₁-C₆-alkyl)CON(C₁-C₆-alkyl-OH)₂; (C₁-C₆-alkyl)CON(C₁-C₆-alkyl-OH—C₁-C₆-alkyl)₂; (C₁-C₆-alkyl)COO(C₁-C₆-alkyl); (C₁-C₆-alkyl)CO₂H; (C₁-C₆-alkyl)OCOO(C₁-C₆-alkyl); (C₁-C₆-alkyl)S(C₁-C₆-alkyl); (C₁-C₆-alkyl)SO(C₁-C₆-alkyl); (C₁-C₆-alkyl)SO₂(C₁-C₆-alkyl); phthalidyl, (5-methyl-2-oxo-1,3-dioxol-4-yl)methyl;

CH(A)SO₃B, wherein A and B are defined as above;

CH(A)COB1, wherein A is defined as above, and B1 is NH₂; NH—C1-C12, more preferably NH—C₁-C₆-alkyl; N—(C1-C12)₂, more preferably N—(C₁-C₆)₂-alkyl;

R₆ is CH(A)CO₂B, wherein A is selected from hydrogen; hydroxyl; C₁-C₁₂-alkyl; C₂-C₁₂-alkenyl; C₂-C₁₂-alkynyl; C₇-C₁₆-arylalkyl, wherein aryl preferably is C₆-C₁₀-aryl and alkyl preferably is C₁-C₆-alkyl; C₃-C₁₀-cycloalkyl; C₄-C₁₆-cycloalkylalkyl, wherein cycloalkyl preferably is C₃-C₁₀-cycloalkyl and alkyl preferably is C₁-C₆-alkyl; C₁-C₆-monohydroxyalkyl; C₂-C₆-dihydroxyalkyl; C₃-C₆-trihydroxyalkyl; C₁-C₆-monoaminoalkyl; C₂-C₆-diaminoalkyl; C₃-C₆-triaminoalkyl; C₁-C₆-alkylguanidino; C₁-C₆-alkylcarboxamide; C₁-C₆-alkylhydroxycarbonyl; C₁-C₆-sulfhydrylalkyl; C₂-C₁₂-alkylthioalkyl, wherein alkylthio is preferably C₁-C₆ and alkyl is preferably C₁-C₆; and wherein B is selected from C₁-C₆-alkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl; phenyl; substituted phenyl; CH₂CH₂N—(C₁-C₆-alkyl)₂; CH₂CON—(C₁-C₆-alkyl)₂.

In an alternative preferred embodiment of the compound according to Embodiment 2, R₁, R₂, R₃, R₄, R₅, and R₆ are defined as above, with the difference that R₂ is not hydrogen.

In a more preferred embodiment of Embodiment 2, the substituents R₁, R₂, R₃, R₄, R₅, and R₆ of compound of Formula (II) have the following meaning:

R₁ is selected from hydrogen; C₁-C₆-alkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

R₂ is selected from hydrogen, C₁-C₆-alkyl; C₁-C₆-monohydroxyalkyl; C₂-C₆-dihydroxyalkyl; C₃-C₆-trihydroxyalkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

R₃ is selected from hydrogen; C₁-C₆-alkyl; C₂-C₆-alkenyl; C₂-C₆-alkynyl, C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl;

R₄ is selected from hydrogen; C₁-C₆-alkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

R₅ is selected from hydrogen, C₁-C₆-alkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl; CH(A)CO₂B, wherein A is selected from hydrogen; C₁-C₆-alkyl; C₇-C₁₆-arylalkyl, wherein aryl preferably is C₆-C₁₀-aryl and alkyl preferably is C₁-C₆-alkyl; C₃-C₁₀-cycloalkyl; C₄-C₁₆-cycloalkylalkyl, wherein cycloalkyl preferably is C₃-C₁₀-cycloalkyl and alkyl preferably is C₁-C₆-alkyl; and wherein B is selected from hydrogen; C₁-C₆-alkyl; C₂-C₆-alkenyl; C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

CH(A)SO₃B, wherein A and B are defined as above;

CH(A)COB1, wherein A is defined as above, and B1 is NH₂;

R₆ is CH(A)CO₂B, wherein A is selected from hydrogen; C₁-C₆-alkyl; C₇-C₁₆-arylalkyl, wherein aryl preferably is C₆-C₁₀-aryl and alkyl preferably is C₁-C₆-alkyl; C₃-C₁₀-cycloalkyl; C₄-C₁₆-cycloalkylalkyl, wherein cycloalkyl preferably is C₃-C₁₀-cycloalkyl and alkyl preferably is C₁-C₆-alkyl; and wherein B is selected from C₁-C₆-alkyl; C₂-C₆-alkenyl; C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl; phenyl; substituted phenyl; CH₂CH₂N—(C₁-C₆-alkyl)₂; CH₂CON—(C₁-C₆-alkyl)₂.

In an alternative more preferred embodiment of the compound according to Embodiment 2,

R₁, R₂, R₃, R₄, R₅, and R₆ are defined as above, with the difference that R₂ is not hydrogen.

In an even more preferred embodiment of Embodiment 2, the substituents R₁, R₂, R₃, R₄, R₅, and R₆ of compound of Formula (II) have the following meaning:

R₁ is selected from hydrogen; C₁-C₆-alkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₇-C₁₆-arylalkyl, wherein aryl preferably is C₆-C₁₀-aryl and alkyl preferably is C₁-C₆-alkyl;

R₂ is selected from hydrogen, C₁-C₆-alkyl; C₂-C₆-alkenyl; C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

R₃ is selected from hydrogen; C₁-C₆-alkyl; C₇-C₁₆-arylalkyl, wherein aryl preferably is C₆-C₁₀-aryl and alkyl preferably is C₁-C₆-alkyl;

R₄ is selected from hydrogen; C₁-C₆-alkyl; C2-C6-alkynyl;

R₅ is selected from hydrogen, C₁-C₆-alkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;

CH(A)CO₂B, wherein A is selected from hydrogen; C₁-C₆-alkyl; C₇-C₁₆-arylalkyl, wherein aryl preferably is C₆-C₁₀-aryl and alkyl preferably is C₁-C₆-alkyl; and wherein B is selected from hydrogen; C₁-C₆-alkyl; C₂-C₆-alkenyl; C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl; phenyl; substituted phenyl;

R₆ is CH(A)CO₂B, wherein A is selected from hydrogen; C₁-C₆-alkyl; C₇-C₁₆-arylalkyl, wherein aryl preferably is C₆-C₁₀-aryl and alkyl preferably is C₁-C₆-alkyl; C₄-C₁₆-cycloalkylalkyl, wherein cycloalkyl preferably is C₃-C₁₀-cycloalkyl and alkyl preferably is C₁-C₆-alkyl; and wherein B is selected from and wherein B is selected from C₁-C₆-alkyl; C₂-C₆-alkenyl; C₂-C₆-alkynyl; C₇-C₁₆-arylalkyl, wherein aryl preferably is C₆-C₁₀-aryl and alkyl preferably is C₁-C₆-alkyl; phenyl substituted phenyl; CH₂CH₂N—(C₁-C₆-alkyl)₂; CH₂CON—(C₁-C₆-alkyl)₂.

In an alternative even more preferred embodiment of the compound according to Embodiment 2, R¹, R², R³, R⁴, R⁵ and R⁶ are defined as above, with the difference that R² is not hydrogen.

Further preferred embodiments of the compound of Embodiment 2 are shown in Table 2-1.

TABLE 2-1
Further preferred embodiments of compounds according to Formula (II)

| | Further preferred | Particularly preferred | Most preferred |
|---|---|---|---|
| R₁ | C1-C6-alkyl, | methyl, ethyl, 2- | methyl, |
| | C1-C6-alkenyl, C₄-C₁₆- | phenylethyl, | cyclopropylmethyl, or |
| | cycloalkylalkyl, or C₇- | cyclopropylmethyl or | allyl |
| | C₁₆-arylalkyl | allyl | |
| R₂ | as defined above for | C1-C6-alkyl, particularly | Methyl, ethyl, benzyl |
| | the even more | methyl, ethyl and | or |
| | preferred embodiment | (substituted) propyl; | 3-phenylpropyl |
| | or the alternative even | arylalkyl as defined | |
| | more preferred | above for the even | |
| | embodiment, with the | more preferred | |
| | preference that R₂ is | embodiment | |
| | not a group which | | |
| | forms an ester with O | | |
| R₃ | hydrogen, | hydrogen, methyl, and | hydrogen |
| | C1-C6-alkyl or C7- | benzyl | |
| | C16-arylalkyl | | |
| R₄ | hydrogen, | hydrogen, methyl or | hydrogen |
| | C1-C6-alkyl or C2-C6- | propargyl | |
| | alkynyl | | |
| R₅ | as defined above for | hydrogen, C1-C6 alkyl, | hydrogen |
| | the even more | C₇-C₁₆-arylalkyl, C₄-C₁₆- | |
| | preferred embodiment | cycloalkylalkyl | |
| R₆ | as defined above for | CH(A)CO₂B, wherein A | CH(A)CO₂B, wherein |
| | the even more | is hydrogen; C1-C6- | A is hydrogen and |
| | preferred embodiment | alkyl; C4-C16- | C1-C6-alkyl, and B is |
| | | cycloalkylalkyl, C₇-C₁₆- | C1-C6-alkyl, |
| | | arylalkyl; and B is C1- | CH₂CH₂N(CH₃)₂, or |
| | | C6-alkyl, phenyl, | CH₂CON—(C₂H₅)₂ |
| | | substituted phenyl, | |
| | | CH₂CH₂N—(C₁-C₆-alkyl)₂ | |
| | | or | |
| | | CH₂CON—(C₁-C₆-alkyl)₂ | |
| Configuration | α, β | α, β | α, β |
| C6 | | | |
| Connectivity | C—C single bond, | C—C single bond | C—C single bond |
| C7-C8 | C—C double bond | | |

All preferred embodiments for compounds according to Formula (II) as described above and in Table 2-1 may be combined with one another as well as with all the preceding and following embodiments of Embodiment 2. Also all alternative embodiments for compounds according to Formula (II) may be combined with one another as well as with all the preceding and following embodiments of Embodiment 2.

In a particularly preferred embodiment, R₁ and R₂ of compounds according to Formula (II) represent alkyl at the same time, in particular methyl. In an alternative particularly preferred embodiment, R₁ represents cycloalkylalkyl, in particular cyclopropylmethyl, and R₂ represents alkyl or arylalkyl, in particular methyl, benzyl or 3-phenylpropyl. Compounds of Formula (II) with this substitution pattern have been found to be particularly suitable for the enhancement of the oral, rectal, transdermal and nasal bioavailability.

The compounds of Formula (II) according to Embodiment 2 may be used for the treatment of pain.

Specifically, the pain which may be treated by the compound of Formula (II) according to Embodiment 2 comprises acute and chronic pain; pain on the locomotor system such as pain in the neck, back, hip, knee, shoulder; arthritic pain, osteoarthritic pain, or myofacial pain; treatment of complex regional pain syndromes, phantom pain, facial neuralgia, postherpetic neuralgia, rheumatalgia, rheumatic pain, sciatic pain, spinal pain, cancer pain, tumor pain, pain from burns, pain after accidents, pain due to acute and chronic inflammation, visceralgia, headaches such as for example tension headaches, cervically related headache or migraine, pain after central lesions such as for example with paraplegia or thalamic lesions, neuralgic pain such as zoster neuralgia, postzoster neuralgia, ischaemic pain such as angina pectoris or peripheral occlusive arterial disease, postoperative pain, neuropathic pain such as pain with diabetic neuropathy, pain after virus infections or pain after nerve lesions, hyperalgesia, allodynia, idiopathic pain, visceral pain, abdominal pain, and toothache.

In particular, the compound of Formula (II) according to Embodiment 2 may be used for the treatment of acute and chronic pain, pain of the locomotor system, arthritic and osteoarthritic pain, cancer and tumor pain, postoperative pain, neuropathic pain, migraine, and inflammatory pain.

The compound of Formula (II) according to Embodiment 2 is further suitable for the treatment of gastric diseases (inflammation of the stomach, gastric ulcers), intestinal diseases, particularly chronic inflammation of the small and large intestines (irritable colon syndrome—colon irritabile, colitis ulcerosa, Morbus Crohn), diarrhea, constipation, ileus, post-operative ileus, opioid-induced bowel dysfunction and other gastrointestinal motility disorders; rheumatic diseases such as rheumatoid arthritis, osteoarthritis, arthrosis, spondylosis, lumbago, lupus erythematosus and spondylarthropathy; tumors and cancer; obesity and overweight; hepatic disorders, liver inflammatory disorders, obesity and overweight.

Moreover, the compound of Formula (II) according to Embodiment 2 is suitable for the withdrawal of drug addiction, such as to opiates, cocaine or alcohol, for the withdrawal of food, buying, internet, computer, phone and gambling addiction, for the treatment of psychic diseases, psychosis, schizophrenia, stress-related conditions (e.g. depression and anxiety), eating disorders and to reduce food intake in humans.

Embodiment 2 also relates to a composition for use in an orally, rectally, transdermally or nasally administered medicament, the composition comprising the compound of Formula (II) and at least one permeation enhancer, which is selected from the group consisting of saturated and/or unsaturated organic fatty acids, or pharmaceutically and pharmacologically acceptable salts or esters thereof; thiomers; and further organic compounds, selected from acetone; alcohols, glycols and glycerides such as ethanol, caprylic alcohol, propylene glycol; essential oils such as niaouli oil, eucalyptus oil, Alpinia oxyphylla oil, turpentine oil, sweet basil oil, tulsi oil, cardamom oil, peppermint oil, fennel oil, black cumin oil; terpenes such as geraniol, nerol, linalool, limonene, α-terpineol, β-terpineol, γ-terpineol, menthol, carveol, menthone, pulegone, iso-pulegone, piperitone, carvomenthone, carvone, 1,8-cineole, α-thujene, car-3-ene, α-pinene, β-pinene, verbenol, verbenone, verbanone, camphor, fenchone, farnesol, nerolidol, (−)-guaiol, (+)-cedrol, (−)-α-bisabolol, bisabolene, azulenes, (+)-longifolene, (−)-isolongifolol, β-caryphyllene, (+)-aromadendrene, (+)-β-cedrene, phytol, squalene, (+)-limonene, (+)-carvone, (+)-neomenthol, β-caryophyllene oxide, (+)-cedryl acetate; pyrrolidones such as 2-pyrrolidone, 1-methyl-2-pyrrolidone, 1-ethyl-2-pyrrolidone, 1-butyl-2-pyrrolidone, 1-hexyl-2-pyrrolidone, 1-octyl-2-pyrrolidone, 1-(2-hydroxyethyl)-2-pyrrolidone; oxazolidinones such as 4-decyloxazolidin-2-one; substituted amino acetates such as dodecyl-N,N-dimethylaminoacetate, dodecyl-2-methyl-2-(N,N-dimethylaminoacetate); azone and derivatives thereof; surfactants such as sodium lauryl sulphate, cetryltrimethyl ammonium bromide, nonoxynol surfactants, dodecyl betaine, sorbitan monolaureate, polysorbates (e.g. 20, 40, 60, 65, 80), dodecyldimethyl ammoniumpropane sulfate; N,N-dimethyformamide; dimethylsulfoxide, decylmethylsulfoxide; phospholipids such as phosphatidyl glycerol derivatives; cyclodextrin and cyclodextrin complexes; amino acid derivatives such as esters; glucosamine; urea and derivatives; polysaccharides, capsaicin; α-tocopherol; liposomes; invasomes, cyclodextrins such as α-, β- and γ-cyclodextrin, methycyclodextrin, hydroxypropyl β-cyclodextrin, dimethyl-β-cyclodextrin; fusidic acid derivatives such as sodium taurodihydrofusidate, sodium glycodihydrofusidate, sodium phosphate-dihydrofusidate; phosphatidylcholine and homologs, didecanoyl-L-α-phosphatidylcholine; bile salts such as sodium cholate, sodium deoxycholate, sodium glycholate, sodium taurocholate, sodium taurodeoxycholate, sodium glycodeoxycholate; starch, degradable starch, soluble starch; dextrane; cellulose; hyaluronic acid esters.

In a preferred embodiment, which may be combined with any of the preceding or following embodiment, the eat least one permeation enhancer is selected from the group consisting of saturated and/or unsaturated organic fatty acids, or pharmaceutically and pharmacologically acceptable salts thereof, and thiomers.

The term “saturated and/or unsaturated organic fatty acid” as used herein has the same meaning as defined in connection with embodiment 1.

Examples for saturated organic fatty acids bearing a terminal carboxylic group and a saturated, linear alkyl chain include behenic acid (C22), arachidic acid (C20), stearic acid (C18), palmitic acid (C16), myristic acid (C14), lauric acid (C12), capric acid (C10), and caprylic acid (C8). Examples for unsaturated organic fatty acids with one terminal carboxylic group and a linear alkyl at least one olefinic bond include erucic acid (C22, cis-Δ¹³), eicosapentaenoic acid (C20, cis,cis,cis,cis,cis-Δ⁵,Δ⁸,Δ¹¹,Δ¹⁴,Δ¹⁷), arachidonic acid (C20, cis,cis,cis,cis-Δ⁵,Δ⁸,Δ¹¹,Δ¹⁴), α-linolenic acid (C18, cis,cis,cis-Δ⁹,Δ¹²,Δ¹⁵), linoelaidic acid (C18, trans,trans-Δ⁹,Δ¹²), linoleic acid (C18, cis,cis-Δ⁹,Δ¹²), vaccenic acid (C18, trans-Δ¹¹), elaidic acid (C18, trans-Δ⁹), oleic acid (C18, cis-Δ⁹), sapienic acid (C16, cis-Δ⁶), palmitoleic acid (C16, cis-Δ⁹) myristoleic acid (C14, cis-Δ⁹).

The aliphatic tail chain of the organic fatty acid may be further substituted such as by alkyl groups, hydroxyl groups and/or carboxyl groups. Examples for saturated and unsaturated fatty acids wherein the aliphatic tail chain bears at least one further substituent are ricinoleic acid and phytanic acid.

The substituents of the aliphatic tail chain of the organic fatty acid may also be further substituted. In particular, hydroxyl groups may be polyalkoxylated, for example by reaction with ethylene oxide to form polyethylene glycol ethers resulting in polyalkoxylated saturated and/or unsaturated organic fatty acids. In a preferred embodiment of the composition of Embodiment 2, which may be combined with any of the preceding and following embodiments, the at least one permeation enhancer (component (b)) is a polyalkoxylated saturated and/or unsaturated organic fatty acid. Particularly preferred in this regard is Cremophor® EL Castor Oil by BASF, wherein the major component is polyethoxylated castor oil, which is derived by reacting castor oil with ethylene oxide in a molar ratio of 1:35.

A pharmaceutically and pharmacologically acceptable salt of the saturated and/or unsaturated organic fatty acid may be derived from acid or base addition to the fatty acid. In a preferred embodiment of the composition of Embodiment 2, which may be combined with any of the preceding or following embodiments, the pharmaceutically and pharmacologically acceptable salt of the saturated and/or unsaturated organic fatty acid is a metal base addition salt, such as a lithium salt, sodium salt, potassium salt, beryllium salt, magnesium salt, and calcium salt. Particularly preferred is a sodium or potassium salt.

The term “thiomer” as used herein has the same meaning as defined in connection with Embodiment 1. Herein, the thiomer is preferably selected from the group consisting of PAA₄₅₀ and PAA₄₅₀-Cys.

It is particularly preferred to provide the compound of Formula (II) in a composition for use in a orally, rectally, transdermally or nasally administered medicament together with at least one permeation enhancer selected from the group consisting of capric acid, or a pharmaceutically and pharmacologically acceptable salt thereof, lauric acid, or a pharmaceutically and pharmacologically acceptable salt thereof, Cremophor® EL, PAA₄₅₀ and PAA₄₅₀-Cys. Preferably, said composition is used as in orally, rectally or nasally administered medicament, and particularly preferably, said composition is used in an orally or rectally administered medicament.

Permeation enhancers, which are particularly suitable for transdermal administration of the composition of Embodiment 2 are selected from acetone; alcohols, glycols and glycerides such as ethanol, caprylic alcohol, propylene glycol; essential oils such as niaouli oil, eucalyptus oil, Alpinia oxyphylla oil, turpentine oil, sweet basil oil, tulsi oil, cardamom oil, peppermint oil, fennel oil, black cumin oil; terpenes such as geraniol, nerol, linalool, limonene, α-terpineol, β-terpineol, γ-terpineol, menthol, carveol, menthone, pulegone, iso-pulegone, piperitone, carvomenthone, carvone, 1,8-cineole, α-thujene, car-3-ene, α-pinene, β-pinene, verbenol, verbenone, verbanone, camphor, fenchone, farnesol, nerolidol, (−)-guaiol, (+)-cedrol, (−)-α-bisabolol, bisabolene, azulenes, (+)-longifolene, (−)-isolongifolol, caryphyllene, (+)-aromadendrene, (+)-β-cedrene, phytol, squalene, (+)-limonene, (+)-carvone, (+)-neomenthol, β-caryophyllene oxide, (+)-cedryl acetate; pyrrolidones such as 2-pyrrolidone, 1-methyl-2-pyrrolidone, 1-ethyl-2-pyrrolidone, 1-butyl-2-pyrrolidone, 1-hexyl-2-pyrrolidone, 1-octyl-2-pyrrolidone, 1-(2-hydroxyethyl)-2-pyrrolidone; oxazolidinones such as 4-decyloxazolidin-2-one; substituted amino acetates such as dodecyl-N,N-dimethylaminoacetate, dodecyl-2-methyl-2-(N,N-dimethylaminoacetate); azone and derivatives thereof; surfactants such as sodium lauryl sulphate, cetryltrimethyl ammonium bromide, nonoxynol surfactants, dodecyl betaine, sorbitan monolaureate, polysorbates (e.g. 20, 40, 60, 65, 80), dodecyldimethyl ammoniumpropane sulfate; N,N-dimethyformamide; dimethylsulfoxide, decylmethylsulfoxide; phospholipids such as phosphatidyl glycerol derivatives; cyclodextrin and cyclodextrin complexes; amino acid derivatives such as esters; glucosamine; urea and derivatives; polysaccharides, capsaicin; α-tocopherol; liposomes; invasomes.

Permeation enhancers, which are particularly suitable for transdermal administration of the composition of Embodiment 2 are selected from cyclodextrins such as α-, β- and γ-cyclodextrin, methycyclodextrin, hydroxypropyl 3-cyclodextrin, dimethyl-β-cyclodextrin; fusidic acid derivatives such as sodium taurodihydrofusidate, sodium glycodihydrofusidate, sodium phosphate-dihydrofusidate; phosphatidylcholine and homologs, didecanoyl-L-α-phosphatidylcholine; bile salts such as sodium cholate, sodium deoxycholate, sodium glycholate, sodium taurocholate, sodium taurodeoxycholate, sodium glycodeoxycholate; starch, degradable starch, soluble starch; dextrane; cellulose; hyaluronic acid esters; mucoadhesive drug delivery systems.

As regards the amounts of the at least one compound of Formula (II) and the at least one permeation enhancer in the composition according to Embodiment 2, the at least one compound of Formula (II) is preferably comprised in an amount of 0.001 to 98% by weight, based on the total volume of the composition (m/v). More preferably, the amount in the composition of Embodiment 2 is 0.01 to 95% by weight, 0.02 to 90% by weight, 0.05 to 80% by weight, 0.06 to 70% by weight, 0.07 to 50% by weight, and 0.09 to 30% by weight, based on the total volume of the composition (m/v). Particularly preferred is an amount of component the compound of Formula (II) of 0.1 to 20% by weight, based on the total volume of the composition (m/v). These preferred embodiments are combinable with any of the preceding and following embodiments.

The amount of the at least one permeation enhancer in the composition according to Embodiment 2 is preferably, and in combination with any of the preceding and following embodiments, 0.01 to 60% by weight, based on the total volume of the composition (m/v). More preferred is an amount of 0.05 to 40% by weight, and 0.1 to 30% by weight, based on the total volume of the composition (m/v). Particularly preferred is an amount of 0.2 to 20% by weight of the at least one permeation enhancer, based on the total volume of the composition (m/v).

The composition according to Embodiment 2 comprises in a preferred embodiment, which may be combined with any of the preceding and following embodiments, at least one further pharmaceutically acceptable excipient. Pharmaceutically acceptable excipients are additional materials used in pharmaceutical compositions in order to bind the pharmaceutically active ingredients into a form suitable for administration. These pharmaceutically acceptable excipients may be selected from the same group as described for embodiment 1 of the present invention.

Embodiment 2 also relates to a pharmaceutical formulation comprising compound of Formula (II) according to Embodiment 2 or the composition of Embodiment 2 as described above.

For the rectal administration of the compound of Formula (II) or the composition according to Embodiment 2, the pharmaceutical formulation according to Embodiment 2 is preferably in the dosage form of a suppository or an enema.

For the sublingual administration of the compound of Formula (II) or the composition according to Embodiment 2, the pharmaceutical formulation according to Embodiment 2 is preferably in the dosage form of a sublingual tablet, a sublingual film, or a sublingual spray.

For the buccal administration of the compound of Formula (II) or the composition according to Embodiment 2, the pharmaceutical formulation according to Embodiment 2 is preferably in the dosage form of a buccal tablet, a buccal patch, a buccal film, a buccal liquid, a buccal semisolid, a buccal spray, or a lollipop.

For the peroral administration of the compound of Formula (II) or the composition according to Embodiment 2, the pharmaceutical formulation according to Embodiment 2 is preferably in the dosage form of a tablet, a pill, a dragée, a capsule, a softgel capsule.

For the transdermal administration of the compound of Formula (II) or the composition according to Embodiment 2, the pharmaceutical formulation according to Embodiment 2 is preferably in the form of a transdermal patch (such as reservoir-type and matrix-type patches), microneedles, sonophoresis, electroporation, electro-osmosis, iontophoresis, iontophoresis patch, short-duration shock waves, photomechanical waves.

For the nasal administration of the compound of Formula (II) or the composition according to Embodiment 2, the pharmaceutical formulation according to Embodiment 2 is preferably in the dosage form of a spray (such as liquid spray, powder spray), squirt system or drops.

In a particularly preferred embodiment, which may be combined with any of the preceding or following embodiments, the aforementioned tablet, pill, dragée, capsule, or softgel capsule has an enteric coating allowing a controlled release of the compound of Formula (II) by targeting target later segments of the gastrointestinal tract by using an enteric coating known in the art that dissolves at higher pH values.

For the transdermal administration of the compound of Formula (II) according to Embodiment 2, the pharmaceutical formulation according to Embodiment 2 is preferably in the dosage form of a transdermal patch (e.g. reservoir-type, matrix-type patches), microneedles, sonophoresis, electroporation, electro-osmosis, iontophoresis, iontophoresis patch, short-duration shock waves, photomechanical waves.

For the nasal administration of the compound of Formula (II) or the composition according to Embodiment 2, the pharmaceutical formulation according to Embodiment 2 is preferably in the dosage form of a spray (e.g. liquid, powder), squirt system or drops.

The pharmaceutical formulation comprising the compound of Formula (II) or the composition according to Embodiment 2 comprises in a preferred embodiment, which may be combined with any of the preceding or following embodiments, 0.1 to 3,000 mg of compound of Formula (II). In a more preferred embodiment, it comprises 1 to 500 mg of compound of Formula (II), in an even more preferred embodiment 1.5 to 300 mg, and in a particularly preferred embodiment, which may be combined with any of the preceding and following embodiments, 2 to 200 mg of compound of Formula (II).

The pharmaceutical formulation comprising the compound according to Formula (II) may be produced in accordance with established procedures known by the person skilled in the art.

Embodiment 2 of the present invention shall be further illustrated be the following examples.

### EXAMPLE 2-1: 6-(AMINO ACID)-MORPHINAN OPIOID RECEPTOR AGONISTS AND THEIR ESTER DERIVATIVES (ESTERIFICATION OF THE AMINO ACID CARBOXYL GROUP)

The c log P and log D values were calculated with MarvinSketch software [http://www.chemaxon.com/products/marvin/marvinsketch/].

Opioid receptor binding was performed as described in: M. Spetea et al., Eur. J. Pharmacol. 2004, 483, 301-308

TABLE 2-2
Opioid receptor activities of agonists and their esters

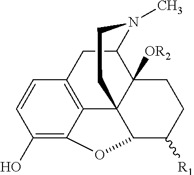

| | R₁ | 6-NH | | Binding Kᵢ (nM)^{b} | | |
|---|---|---|---|---|---|---|
| Compound | (AA) | α/β | R₂ | MOR | DOR | KOR |
| Morphine | OH | — | H | 6.55 | 217 | 113 |
| OMO | O | — | H | 0.97 | 80.5 | 61.6 |
| 14-OMO | O | — | Me | 0.10 | 4.80 | 10.2 |
| HS730 | Gly | α | Me | 0.89 | 15.4 | 43.2 |
| HS730-Et ester | Gly-ester | α | Me | 0.28 | 0.96 | 8.57 |
| HS730-t-Bu ester | Gly-ester | α | Me | 0.48 | 0.54 | 12.1 |
| HS730-nOc ester | Gly-ester | α | Me | 1.29 | 3.23 | 2.97 |
| HS730-benzyl ester | Gly-ester | α | Me | 0.19 | 0.91 | 0.47 |
| HS731 | Gly | β | Me | 0.83 | 7.86 | 44.8 |
| HS731-Et ester | Gly-ester | β | Me | 0.74 | 0.81 | 21.9 |
| HS731-t-Bu ester | Gly-ester | β | Me | 1.30 | 0.68 | 56.2 |
| HS731-nOc ester | Gly-ester | β | Me | 2.56 | 2.71 | 3.87 |
| HS731-benzyl ester | Gly-ester | β | Me | 0.29 | 0.74 | 2.86 |
| 1 | L-Ala | α | Me | 0.77 | 26.9 | 142 |
| 1-t-Bu ester | L-Ala-ester | α | Me | 0.29 | 0.41 | 11.3 |
| 2 | L-Ala | β | Me | 1.90 | 7.71 | 63.7 |
| 2-t-Bu ester | L-Ala-ester | β | Me | 1.13 | 1.36 | 93.6 |
| 3 | L-Phe | α | Me | 0.95 | 3.67 | 28.5 |
| 3-t-Bu ester | L-Phe-ester | α | Me | 0.50 | 0.93 | 32.5 |
| 4 | L-Phe | β | Me | 2.58 | 1.03 | 151 |
| 4-t-Bu ester | L-Phe-ester | β | Me | 2.56 | 2.66 | 151 |
| 5 | Gly | α | Et | 0.57 | 10.3 | 45.2 |
| 5-t-Bu ester | Gly-ester | α | Et | 0.20 | 0.31 | 15.0 |
| 6 | Gly | β | Et | 0.95 | 5.31 | 102 |
| 6-t-Bu ester | Gly-ester | β | Et | 0.81 | 0.57 | 34.1 |
| 7 | Gly | α | PP | 0.19 | 0.22 | 0.73 |
| 7-t-Bu ester | Gly-ester | α | PP | 1.40 | 1.01 | 1.26 |
| 7-Et ester | Gly-ester | α | PP | 0.43 | 0.25 | 0.44 |
| 8 | Gly | β | PP | 0.16 | 0.19 | 0.81 |
| 8-t-Bu ester | Gly-ester | β | PP | 1.03 | 0.92 | 2.02 |
| 8-Et ester | Gly-ester | β | PP | 0.37 | 0.21 | 0.41 |
| 9 | L-Tyr | α | Me | 0.83 | 2.18 | 39.5 |
| 9-t-Bu ester | L-Tyr-ester | α | Me | 0.59 | 0.42 | 12.3 |
| 10 | L-Tyr | β | Me | 3.20 | 3.89 | 186 |
| 10-t-Bu ester | L-Tyr-ester | β | Me | 1.53 | 1.88 | 184 |
| 15 | GABA | α | Me | 0.77 | 12.5 | 45.6 |
| 15-t-Bu ester | GABA-ester | α | Me | 0.22 | 7.99 | 8.82 |
| 16 | GABA | β | Me | 1.41 | 6.61 | 147 |
| 16-t-Bu ester | GABA-ester | β | Me | 0.54 | 1.63 | 8.79 |
| 17 | L-Gln | α | Me | 3.24 | 5.13 | 351 |
| 17-t-Bu ester | L-Gln-ester | α | Me | 0.18 | 0.18 | 8.97 |
| 18 | L-Gln | β | Me | 2.48 | 4.87 | 290 |
| 18-t-Bu ester | L-Gln-ester | β | Me | 1.65 | 1.35 | 163 |
| 19 | L-Glu | α | Me | 1.45 | 9.03 | 87.2 |
| 19-t-Bu ester | L-Glu-ester | α | Me | 0.27 | 0.32 | 18.3 |
| 20 | L-Glu | β | Me | 11.6 | 7.64 | 1252 |
| 20-t-Bu ester | L-Glu-ester | β | Me | 1.53 | 0.19 | 81.5 |
| 21 | L-β-Ala | α | Me | 1.30 | 60.0 | 182 |
| 21-t-Bu ester | L-β-Ala-ester | α | Me | 0.31 | 0.83 | 13.5 |
| 22 | L-β-Ala | β | Me | 1.04 | 13.9 | 71.4 |
| 22-t-Bu ester | L-β-Ala-ester | β | Me | 0.35 | 0.23 | 7.54 |
| 23 | L-Met | α | Me | 0.93 | 4.03 | 109 |
| 23-t-Bu ester | L-Met-ester | α | Me | 0.32 | 0.12 | 10.5 |
| 24 | L-Met | β | Me | 3.88 | 2.40 | 468 |
| 24-t-Bu ester | L-Met-ester | β | Me | 1.83 | 0.81 | 166 |
| 26 | L-Asn | α | Me | 1.17 | 3.37 | 74.0 |
| 26-t-Bu ester | L-Asn-ester | α | Me | 0.45 | 0.33 | 5.33 |
| 27 | L-Asn | β | Me | 1.26 | 2.25 | 103 |
| 27-t-Bu ester | L-Asn-ester | β | Me | 0.60 | 0.66 | 75.8 |
| 28 | L-Val | α | Me | 3.16 | 3.91 | 325 |
| 28-t-Bu ester | L-Val-ester | α | Me | 0.25 | 0.18 | 10.5 |
| 29 | L-Val | β | Me | 3.04 | 3.52 | 305 |
| 29-t-Bu ester | L-Val-ester | β | Me | 1.03 | 0.86 | 214 |
| 30 | D-Val | α | Me | 1.70 | 1.93 | 202 |
| 30-t-Bu ester | D-Val-ester | α | Me | 0.11 | 0.093 | 22.7 |
| 31 | D-Val | β | Me | 1.02 | 1.68 | 159 |
| 31-t-Bu ester | D-Val | β | Me | 1.98 | 0.21 | 206 |
| 32 | L-Val-L-Tyr | α | Me | 0.82 | 1.19 | 69.0 |
| 32-t-Bu ester | L-Val-L-Tyr- | α | Me | 0.11 | 0.17 | 3.50 |
| | ester | | | | | |
| 33 | L-Val-L-Tyr | β | Me | 0.44 | 1.38 | 390 |
| 33-t-Bu ester | L-Val-L-Tyr- | β | Me | 0.14 | 0.27 | 24.1 |
| | ester | | | | | |
| 34 | L-Thr | α | Me | 1.03 | 4.13 | 120 |
| 34-t-Bu ester | L-Thr-ester | α | Me | 0.20 | 0.37 | 9.24 |
| 35 | L-Thr | β | Me | 0.79 | 5.16 | 58.6 |
| 35-t-Bu ester | L-Thr-ester | β | Me | 0.51 | 0.65 | 102 |
| 36 | L-Ser | α | Me | 2.21 | 5.32 | 196 |
| 36-t-Bu ester | L-Ser-ester | α | Me | 0.20 | 0.17 | 6.20 |
| 37 | L-Ser | β | Me | 2.14 | 5.29 | 152 |
| 37-t-Bu ester | L-Ser-ester | β | Me | 0.60 | 0.64 | 83.3 |
| 38 | L-Lys | α | Me | 0.19 | 1.27 | 12.6 |
| 38-t-Bu ester | L-Lys-ester | α | Me | 0.17 | 0.092 | 0.89 |
| 39 | L-Lys | β | Me | 0.53 | 3.34 | 33.7 |
| 39-t-Bu ester | L-Lys-ester | β | Me | 0.58 | 0.88 | 21.4 |
| 40 | L-Leu | α | Me | 0.68 | 2.34 | 141 |
| 40-t-Bu ester | L-Leu-ester | α | Me | 0.087 | 0.13 | 8.07 |
| 41 | L-Leu | β | Me | 1.32 | 1.01 | 297 |
| 41-t-Bu ester | L-Leu-ester | β | Me | 0.57 | 0.42 | 240 |
| 42 | L-Ile | α | Me | 0.84 | 3.20 | 131 |
| 42-t-Bu ester | L-Ile-ester | α | Me | 1.04 | 1.00 | 29.2 |
| 43 | L-Ile | β | Me | 1.46 | 1.30 | 163 |
| 43-t-Bu ester | L-Ile-ester | β | Me | 2.87 | 2.12 | 208 |
| 44 | L-Asp | α | Me | 1.36 | 14.6 | 50.2 |
| 44-t-Bu ester | L-Asp-ester | α | Me | 0.40 | 0.20 | 12.6 |
| 45 | L-Asp | β | Me | 3.42 | 22.6 | 351 |
| 45-t-Bu ester | L-Asp-ester | β | Me | 2.14 | 0.40 | 172 |
| 46 | L-Trp | β | Me | 0.65 | 1.19 | 8.66 |
| 46-t-Bu ester | L-Trp-ester | β | Me | 0.26 | 0.36 | 9.89 |

^{a}Determined in in vitro binding assays in rat brain (MOR and DOR) or guinea pig brain (KOR) membranes; AA: amino acid residue; OMO: oxymorphone; 14-OMO: 14-O-methyloxymorphone; PP: phenylpropyl; morphine, OMO and 14-OMO are reference compounds.

All 6-(amino acid)-morphinan esters of compounds 1-10, 15-24, 24-46 and HS730 and HS731 show high binding affinities to the MOR (Kᵢ values in the low nanomolar and subnanomolar range). Some of the esters display comparable binding affinities to both DOR and MOR while for others the DOR binding is somewhat lower. All esters show lower KOR binding compared to binding to the MOR and DOR.

### EXAMPLE 2-2: AGONIST POTENCIES OF HS731 IN THE MOUSE VAS DEFERENS PREPARATION IN COMPARISON TO ITS ETHYL ESTER (HS731-ET ESTER)

The mouse vas deferens bioassay was performed as described in: M. Spetea et al., Eur. J. Pharmacol. 2004, 483, 301-308

TABLE 2-3
Agonist potencies of HS731 in the mouse vas deferens
preparation in comparison to its ethyl ester (HS731-Et ester)

| Compound | IC₅₀ (nM) |
|---|---|
| DAMGO | 76.6 |
| 14-OMO | 7.76 |
| HS731 | 7.00 |
| HS731-Et ester | 23.8 |

14-OMO: 14-O-methyloxymorphone;
DAMGO and 14-OMO are reference compounds.

In mouse vas deferens bioassay, HS731 exhibits a similar high agonist potency compared to its parent compound 14-O-methyloxymorphone, while the ethyl ester of HS731 (HS731-Et ester) is about 3.5 times less potent than HS731.

### EXAMPLE 2-3: ANTINOCICEPTIVE POTENCIES OF HS731 AND ITS ETHYL ESTER (HS731-ET ESTER) IN THE TAIL-FLICK TEST IN RATS AFTER ORAL ADMINISTRATION

The rat tail-flick test was performed as described in: S. Furst et. al. J. Pharmacol. Exp. Ther. 2005, 312, 609-618

TABLE 2-4
Antinociceptive potencies of HS731 and its ethyl ester (HS731-
Et ester) in the tail-flick test in rats after oral administration

| | ED₅₀ (p.o., mg/kg) | | | | |
|---|---|---|---|---|---|
| Compound | 30 min | 1 h | 2 h | 3 h | 4 h |
| HS731 | 21.0 | 5.52 | 6.78 | 6.33 | |
| HS731-Et ester | >2.5 | 2.34 | 0.85 | 0.71 | 0.82 |

Surprisingly, the antinociceptive potency of the bioreversible ethyl ester of HS731 (HS731-Et ester) was considerably higher than the parent compound HS731 after oral administration—after 1 h ca. 2-fold, after 2 h ca. 8-fold and after 3 h ca. 9-fold higher. Thus, the oral bioavailability of the bioreversible ethyl ester derivative HS731-Et ester is significantly better than of the parent compound HS731. Despite the fact that in the in vitro mouse vas deferens bioassay, the HS731-Et ester is much less potent than HS731 as an agonist, in vivo it is surprisingly a highly potent agonist in producing an analgesic effect in rats after oral administration.

### EXAMPLE 2-4: PHYSIOCHEMICAL PROPERTIES OF BIOREVERSIBLE ESTERS OF OPIOID AGONISTS

The c log P and log D values were calculated with MarvinSketch software [http://www.chemaxon.com/products/marvin/marvinsketch/].

TABLE 2-5
Physiochemical properties of bioreversible esters of opioid agonists

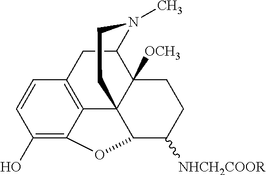

| R | clogP | clogD |
|---|---|---|
| C₂H₅ | 1.43 | 0.36 |
| C(CH₃)₃ | 2.12 | 1.06 |
| (CH₂)₇CH₃ | 4.17 | 3.11 |
| Ph | 2.73 | 1.66 |
| CH₂Ph | 2.79 | 1.73 |
| CH₂CON(C₂H₅)₂ | 0.90 | −0.15 |
| CH₂N(CH₃)₂ | 1.20 | 0.13 |
| CH₂OCOOC₂H₅ | 1.91 | 0.84 |
| CH₂OCO—C(CH₃)₃ | 2.73 | 1.66 |

Surprisingly, it was found that all bioreversible esters listed in Table 2-5 exhibit c log P and c log P values which are well suitable for high bioavailability after oral, rectal, transdermal and/or nasal administration.

### EXAMPLE 2-5: 6-(AMINO ACID)-MORPHINAN OPIOID RECEPTOR ANTAGONISTS AND THEIR T-BUTYL ESTER DERIVATIVES (ESTERIFICATION OF THE AMINO ACID CARBOXYL GROUP)

The c log P and log D values were calculated with MarvinSketch software [http://www.chemaxon.com/products/marvin/marvinsketch/].

Opioid receptor binding was performed as described in: M. Spetea et al., Eur. J. PharmacoL 2004, 483, 301-308

TABLE 2-6
Opioid receptor activities of antagonists and their t-butyl esters

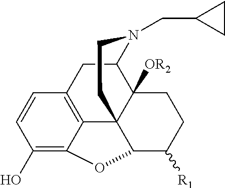

| | R₁ | | | Binding Kᵢ (nM)^{b} | | |
|---|---|---|---|---|---|---|
| Compound | (AA) | 6-NH α/β | R₂ | MOR | DOR | KOR |
| NTX | O | — | H | 0.54 | 14.8 | 0.81 |
| 14-OMe-NTX | O | — | Me | 0.46 | 7.88 | 1.17 |
| 14-OEt-NTX | O | — | Et | 0.35 | 4.13 | 0.93 |
| 55 | L-Phe | α | Et | 0.35 | 0.70 | 2.60 |
| 55-tBu ester | L-Phe-ester | α | Et | 0.63 | 2.38 | 4.12 |
| 56 | L-Phe | β | Et | 1.04 | 1.76 | 11.6 |
| 56-tBu ester | L-Phe-ester | β | Et | 2.00 | 6.33 | 17.9 |
| 57 | L-Phe | α | Me | 0.51 | 1.17 | 4.21 |
| 57-tBu ester | L-Phe-ester | α | Me | 0.68 | 0.58 | 1.78 |
| 58 | L-Phe | β | Me | 1.23 | 2.02 | 8.42 |
| 58-tBu ester | L-Phe-ester | β | Me | 2.41 | 4.56 | 13.1 |
| 59 | L-Phe | α | H | 1.41 | 3.53 | 2.65 |
| 59-tBu ester | L-Phe-ester | α | H | 1.00 | 2.15 | 5.00 |
| 60 | L-Phe | β | H | 1.83 | 2.79 | 8.99 |
| 60-tBu ester | L-Phe-ester | β | H | 5.49 | 1.12 | 17.6 |
| 61 | Gly | α | Me | 0.68 | 5.15 | 4.65 |
| 61-tBu ester | Gly-ester | α | Me | 0.43 | 0.50 | 1.41 |
| 62 | Gly | β | Me | 1.31 | 4.76 | 9.31 |
| 62-Bu ester | Gly-ester | β | Me | 2.66 | 0.96 | 8.36 |
| 63 | L-β-Ala | α | Me | 1.35 | 6.25 | 7.81 |
| 63-tBu ester | L-β-Ala | α | Me | 0.81 | 2.10 | 1.41 |
| 64 | L-β-Ala | β | Me | 1.51 | 8.12 | 11.7 |
| 64-tBu ester | L-β-Ala | β | Me | 1.52 | 0.91 | 2.13 |
| 65 | GABA | α | Me | 1.21 | 10.7 | 5.31 |
| 65-tBu ester | GABA-ester | α | Me | 0.46 | 2.69 | 0.60 |
| 66 | GABA | β | Me | 3.36 | 10.2 | 16.8 |
| 66-tBu ester | GABA-ester | β | Me | 2.13 | 3.78 | 2.81 |
| 68 | L-Val | α | Me | 0.44 | 0.53 | 3.34 |
| 68-tBu ester | L-Val-ester | α | Me | 1.81 | 0.85 | 3.13 |
| 69 | L-Val | β | Me | 0.66 | 1.35 | 3.00 |
| 69-tBu ester | L-Val-ester | β | Me | 1.23 | 0.70 | 4.32 |
| 70 | D-Val | α | Me | 0.62 | 0.71 | 3.54 |
| 70-tBu ester | D-Val-ester | α | Me | 0.63 | 0.51 | 18.8 |
| 71 | D-Val | β | Me | 1.52 | 0.35 | 23.0 |
| 71-tBu ester | D-Val-ester | β | Me | 1.94 | 0.24 | 15.2 |
| 72 | L-Ala | β | Me | 2.13 | 5.80 | 11.5 |
| 72-tBu ester | L-Ala-ester | β | Me | 0.89 | 0.32 | 9.68 |
| 73 | L-Asp | β | Me | 2.24 | 0.64 | 11.7 |
| 73-tBu ester | L-Asp | β | Me | 4.41 | 1.20 | 18.2 |
| 74 | L-Glu | α | Me | 1.93 | 3.61 | 6.66 |
| 74-tBu ester | L-Glu-ester | α | Me | 1.93 | 0.49 | 10.4 |
| 75 | L-Glu | β | Me | 3.35 | 2.77 | 12.9 |
| 75-tBu ester | L-Glu-ester | β | Me | 3.34 | 1.50 | 11.6 |
| 76 | L-Leu | α | Me | 0.72 | 2.20 | 7.87 |
| 76-tBu ester | L-Leu | α | Me | 0.13 | 0.092 | 0.80 |
| 77 | L-Leu | β | Me | 1.96 | 1.13 | 11.0 |
| 77-tBu ester | L-Leu | β | Me | 0.13 | 0.094 | 0.68 |
| 78 | L-Ile | β | Me | 1.62 | 1.44 | 9.49 |
| 78-tBu ester | L-Ile | β | Me | 0.98 | 0.52 | 5.67 |

^{a}Calculated with MarvinSketch software; ^{b}Determined in in vitro binding assays in rat brain (MOR and DOR) or guinea pig brain (KOR) membranes; AA: amino acid residue; NTX: Naltrexone; 14-OMe-NTX: 14-O-Methylnaltrexone; 14-OEt-NTX: 14-O-Ethylnaltrexone; NTX, 14-OMe-NTX und 14-OEt-NTX are reference compounds.

All 6-(amino acid)-morphinan t-butyl esters of compounds 55-66 and 68-78 show high binding affinities to the MOR (Kᵢ values in the low nanomolar and subnanomolar range). Some of the esters display comparable binding affinities to both DOR and MOR while for others the DOR binding is somewhat lower. All esters show lower KOR binding compared to binding to the MOR and DOR.

### EXAMPLE 2-6: PHYSIOCHEMICAL PROPERTIES OF BIOREVERSIBLE ESTERS OF OPIOID ANTAGONISTS

The c log P and log D values were calculated with MarvinSketch software [http://www.chemaxon.com/products/marvin/marvinsketch/].

TABLE 2-7
Physiochemical properties of bioreversible esters of opioid antagonists

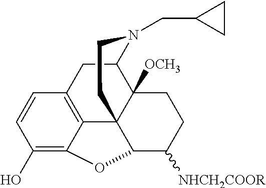

| R | clogP | clogD |
|---|---|---|
| C₂H₅ | 2.21 | 1.50 |
| C(CH₃)₃ | 2.90 | 2.19 |
| (CH₂)₇CH₃ | 4.95 | 4.24 |
| Ph | 3.51 | 2.80 |
| CH₂Ph | 3.58 | 2.86 |
| CH₂CON(C₂H₅)₂ | 1.68 | 0.98 |
| CH₂N(CH₃)₂ | 1.98 | 1.27 |
| CH₂OCOOC₂H₅ | 2.69 | 1.98 |
| CH₂OCO—C(CH₃)₃ | 3.51 | 2.80 |

Surprisingly, it was found that all bioreversible esters listed in Table 2-7 exhibit c log P and c log D values which are well suitable for high bioavailability after oral, rectal, transdermal and/or nasal administration.

1. Composition for use in an orally, rectally, transdermally or nasally administered medicament, the composition comprising:
(a) at least one compound of Formula (I) 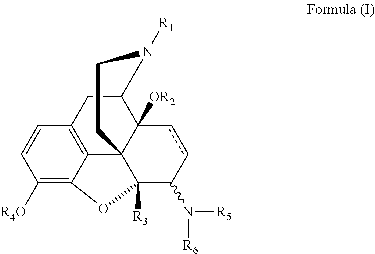
wherein the substituents R₁, R₂, R₃, R₄, R₅ and R₆ have the following meaning:
R₁ is selected from hydrogen; C₁-C₆-alkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;
R₂ is selected from hydrogen, C₁-C₆-alkyl; C₁-C₆-monohydroxyalkyl; C₂-C₆-dihydroxyalkyl; C₃-C₆-trihydroxyalkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;
R₃ is selected from hydrogen; C₁-C₆-alkyl; C₂-C₆-alkenyl; C₂-C₆-alkynyl, C₇-C₁₆-arylalkyl, wherein aryl preferably is C₆-C₁₀-aryl and alkyl preferably is C₁-C₆-alkyl; C₆-C₁₆-arylalkenyl, wherein aryl preferably is C₆-C₁₀-aryl and alkenyl preferably is C₂-C₆-alkenyl;
R₄ is selected from hydrogen; C₁-C₆-alkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl;
R₅ and R₆, which can be the same or different, provided that not both are hydrogen, and are selected from hydrogen, C₁-C₆-alkyl; C₂-C₆-alkenyl; C₂-C₆-alkynyl; C₄-C₁₆-cycloalkylalkyl, wherein cycloalkyl preferably is C₃-C₁₀-cycloalkyl and alkyl preferably is C₁-C₆-alkyl; C₅-C₁₆-cycloalkylalkenyl, wherein cycloalkyl preferably is C₃-C₁₀-cycloalkyl and alkenyl preferably is C₂-C₆-alkenyl; C₅-C₁₆-cycloalkylalkynyl, wherein cycloalkyl preferably is C₃-C₁₀-cycloalkyl and alkynyl preferably is C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl; CH(A)CO₂B, wherein A is selected from hydrogen; C₁-C₆-alkyl; C₇-C₁₆-arylalkyl, wherein aryl preferably is C₆-C₁₀-aryl and alkyl preferably is C₁-C₆-alkyl; C₃-C₁₀-cycloalkyl; C₄-C₁₆-cycloalkylalkyl, wherein cycloalkyl preferably is C₃-C₁₀-cycloalkyl and alkyl preferably is C₁-C₆-alkyl; and wherein B is selected from hydrogen; C₁-C₆-alkyl; C₂-C₆-alkenyl; C₂-C₆-alkynyl; C₇-C₁₆-arylalkyl, wherein aryl preferably is C₆-C₁₀-aryl and alkyl preferably is C₁-C₆-alkyl; C₈-C₁₆-arylalkenyl, wherein aryl preferably is C₆-C₁₀-aryl and alkenyl preferably is C₂-C₆-alkenyl; C₈-C₁₆-arylalkynyl, wherein aryl preferably is C₆-C₁₀-aryl and alkynyl preferably is C₂-C₆-alkynyl; phenyl; substituted phenyl; (C₁-C₆-alkyl)CONH₂;
CH(A)SO₃B, wherein A and B are defined as above;
CH(A)COB1, wherein A is defined as above, and B1 is NH₂;
preferably wherein R₅ is as defined above and
R₆ is CH(A)CO₂H, wherein A is selected from hydrogen; C₁-C₆-alkyl; C₇-C₁₆-arylalkyl, wherein aryl preferably is C₆-C₁₀-aryl and alkyl preferably is C₁-C₆-alkyl; C₃-C₁₀-cycloalkyl; C₄-C₁₆-cycloalkylalkyl, wherein cycloalkyl preferably is C₃-C₁₀-cycloalkyl and alkyl preferably is C₁-C₆-alkyl.
and/or a pharmaceutically acceptable acid addition or base addition salt thereof,
wherein the composition further comprises
(b) at least one permeation enhancer, selected from the group consisting of saturated and/or unsaturated organic fatty acids, or pharmaceutically and pharmacologically acceptable salts or esters thereof; thiomers; and further organic compounds, selected from acetone; alcohols, glycols and glycerides such as ethanol, caprylic alcohol, propylene glycol; essential oils such as niaouli oil, eucalyptus oil, Alpinia oxyphylla oil, turpentine oil, sweet basil oil, tulsi oil, cardamom oil, peppermint oil, fennel oil, black cumin oil; terpenes such as geraniol, nerol, linalool, limonene, α-terpineol, β-terpineol, γ-terpineol, menthol, carveol, menthone, pulegone, iso-pulegone, piperitone, carvomenthone, carvone, 1,8-cineole, α-thujene, car-3-ene, α-pinene, β-pinene, verbenol, verbenone, verbanone, camphor, fenchone, farnesol, nerolidol, (−)-guaiol, (+)-cedrol, (−)-α-bisabolol, bisabolene, azulenes, (+)-longifolene, (−)-isolongifolol, β-caryphyllene, (+)-aromadendrene, (+)-β-cedrene, phytol, squalene, (+)-limonene, (+)-carvone, (+)-neomenthol, β-caryophyllene oxide, (+)-cedryl acetate; pyrrolidones such as 2-pyrrolidone, 1-methyl-2-pyrrolidone, 1-ethyl-2-pyrrolidone, 1-butyl-2-pyrrolidone, 1-hexyl-2-pyrrolidone, 1-octyl-2-pyrrolidone, 1-(2-hydroxyethyl)-2-pyrrolidone; oxazolidinones such as 4-decyloxazolidin-2-one; substituted amino acetates such as dodecyl-N,N-dimethylaminoacetate, dodecyl-2-methyl-2-(N,N-dimethylaminoacetate); azone; surfactants such as sodium lauryl sulphate, cetryltrimethyl ammonium bromide, nonoxynol surfactants, dodecyl betaine, sorbitan monolaureate, polysorbates (e.g. 20, 40, 60, 65, 80), dodecyldimethyl ammoniumpropane sulfate; N,N-dimethyformamide; dimethylsulfoxide, decylmethylsulfoxide; phospholipids such as phosphatidyl glycerol derivatives; cyclodextrin and cyclodextrin complexes; amino acid derivatives such as esters; glucosamine; urea and derivatives; polysaccharides, capsaicin; α-tocopherol; liposomes; invasomes, cyclodextrins such as α-, β- and γ-cyclodextrin, methycyclodextrin, hydroxypropyl β-cyclodextrin, dimethyl-β-cyclodextrin; fusidic acid derivatives such as sodium taurodihydrofusidate, sodium glycodihydrofusidate, sodium phosphate-dihydrofusidate; phosphatidylcholine, didecanoyl-L-α-phosphatidylcholine; bile salts such as sodium cholate, sodium deoxycholate, sodium glycholate, sodium taurocholate, sodium taurodeoxycholate, sodium glycodeoxycholate; starch, degradable starch, soluble starch; dextrane; cellulose; hyaluronic acid esters, as well as mixtures thereof.

2. Composition according to claim 1, wherein it comprises compound (a) in an amount of 0.001 to 98% by weight, based on the total volume of the composition.

3. Composition according to claim 1, wherein the at least one permeation enhancer (b) is selected from the group consisting of saturated and/or unsaturated organic fatty acids, or pharmaceutically and pharmacologically acceptable salts or esters thereof, and thiomers.

4. Composition according to claim 1, wherein the at least one permeation enhancer (b) is selected from the group consisting of capric acid, or a pharmaceutically and pharmacologically acceptable salt thereof, lauric acid, or a pharmaceutically and pharmacologically acceptable salt thereof, Cremophor® EL, PAA₄₅₀ and PAA₄₅₀-Cys.

5. Composition according to claim 1, wherein it comprises the at least one permeation enhancer (b) in an amount of 0.01 to 60% by weight, based on the total volume of the composition.

6. Composition according to claim 1, wherein it comprises at least one pharmaceutically acceptable excipient.

7. Composition according to claim 1, for the treatment of pain.

8. Composition according to claim 1, for the treatment of gastric diseases (inflammation of the stomach, gastric ulcers), intestinal diseases, particularly chronic inflammation of the small and large intestines (irritable colon syndrome—colon irritabile, colitis ulcerosa, Morbus Crohn), diarrhea, constipation and ileus; rheumatic diseases such as rheumatoid arthritis, osteoarthritis, arthrosis, spondylosis, lumbago, lupus erythematosus and spondylarthropathy; tumors and cancer; obesity and overweight; hepatic disorders, and liver inflammatory disorders.

9. Composition according to claim 1, for the withdrawal of drug addiction, such as to opiates, cocaine, alcohol, for the withdrawal of food, buying, Internet, computer, phone and gambling addiction, for the treatment of psychic diseases, psychosis, schizophrenia, stress-related conditions (such as depression and anxiety), eating disorders and for the reduction of food intake in humans.

10. Pharmaceutical formulation wherein it comprises the composition of claim 1.

11. Pharmaceutical formulation according to claim 10, wherein it is in the dosage form of an oral strip, a solution, a softgel, a suspension, an emulsion, a syrup, an elixir, a hydrogel, an adhesive gel, a suppository, an enema, a sublingual tablet, a sublingual film, a sublingual spray, a buccal tablet, a buccal patch, a buccal film, a buccal liquid, a buccal semisolid, a buccal spray, a lollipop.

12. Pharmaceutical formulation according to claim 11, wherein it is in the dosage form of a tablet, a pill, a dragée, a capsule, a softgel capsule.

13. Pharmaceutical formulation according to claim 12, wherein it further comprises an enteric coating.

14. Compound of Formula (II) for use in an orally, rectally, transdermally or nasally administered medicament, 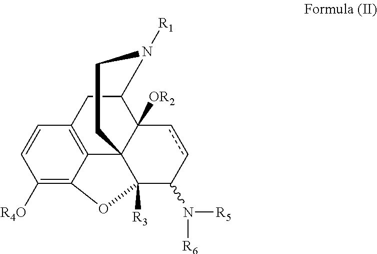
wherein the substituents R₁, R₂, R₃, R₄, R₅ and R₆ have the following meaning:
R₁ is selected from hydrogen; C1-C30, preferably C1-C12, more preferably C₁-C₆-alkyl; C2-C30, preferably C2-C12, more preferably C₂-C₆-alkenyl; C2-C30, preferably C2-C12, more preferably C₂-C₆-alkynyl; C1-C30, preferably C1-C12, more preferably C₁-C₆-monohydroxyalkyl; C2-C30, preferably C2-C12, more preferably C₂-C₆-dihydroxyalkyl; C3-C30, preferably C3-C12, more preferably C₃-C₆-trihydroxyalkyl; C4-C30, preferably C₄-C₁₆-cycloalkylalkyl, wherein cycloalkyl preferably is C₃-C₁₀-cycloalkyl and alkyl preferably is C₁-C₆-alkyl; C5-C30, preferably C₅-C₁₆-cycloalkylalkenyl, wherein cycloalkyl preferably is C₃-C₁₀-cycloalkyl and alkenyl preferably is C₂-C₆-alkenyl; C5-C30, preferably C₅-C₁₆-cycloalkylalkynyl, wherein cycloalkyl preferably is C₃-C₁₀-cycloalkyl and alkynyl preferably is C₂-C₆-alkynyl; C7-C30, preferably C₇-C₁₅-arylalkyl, wherein aryl preferably is C₆-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; C8-C30, preferably C₈-C₁₆-arylalkynyl, wherein aryl preferably is C₆-C₁₀-aryl and alkynyl preferably is C₂-C₆-alkynyl;
R₂ is selected from hydrogen; C1-C30, preferably C1-C12, more preferably C₁-C₆-alkyl; C1-C30, preferably C1-C12, more preferably C₁-C₆-monohydroxyalkyl; C2-C30, preferably C2-C12, more preferably C₂-C₆-dihydroxyalkyl; C3-C30, preferably C3-C12, more preferably C₃-C₆-trihydroxyalkyl; C2-C30, preferably C2-C12, more preferably C₂-C₆-alkenyl; C2-C30, preferably C2-C12, more preferably C₂-C₆-alkynyl; C4-C30, preferably C₄-C₁₆-cycloalkylalkyl, wherein cycloalkyl preferably is C₃-C₁₀-cycloalkyl and alkyl preferably is C₁-C₆-alkyl; C5-C30, preferably C₅-C₁₆-cycloalkylalkenyl, wherein cycloalkyl preferably is C₃-C₁₀-cycloalkyl and alkenyl preferably is C₂-C₆-alkenyl; C5-C30, preferably C₅-C₁₆-cycloalkylalkynyl, wherein cycloalkyl preferably is C₃-C₁₀-cycloalkyl and alkynyl preferably is C₂-C₆-alkynyl; C7-C30, preferably C₇-C₁₆-arylalkyl, wherein aryl preferably is O₆-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; C8-C30, preferably C₈-C₁₆-arylalkynyl, wherein aryl preferably is C₆-C₁₀-aryl and alkynyl preferably is C₂-C₆-alkynyl; C2-C30, preferably C2-C12, more preferably C₂-C₆-alkanoyl; C3-C30, preferably C3-C12, more preferably C₃-C₆-alkenoyl; C3-C30, preferably C3-C12, more preferably C₃-C₆-alkinoyl; C7-C30, preferably C₇-C₁₆-arylalkanoyl, wherein aryl preferably is C₆-C₁₀-aryl and alkanoyl preferably is C₁-C₆-alkanoyl; C9-C30, preferably C₉-C₁₆-arylalkenoyl, wherein aryl preferably is C₆-C₁₀-aryl and alkenoyl preferably is C₃-C₆-alkenoyl; C9-C30, preferably C₉-C₁₆-arylalkinoyl, wherein aryl preferably is C₆-C₁₀-aryl and alkinoyl preferably is C₃-C₆-alkinoyl;
R₃ is selected from hydrogen; C1-C30, preferably C1-C12, more preferably C₁-C₆-alkyl; C2-C30, preferably C2-C12, more preferably C₂-C₆-alkenyl; C7-C30, preferably C₇-C₁₆-arylalkyl, wherein aryl preferably is C₆-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; alkoxyalkyl, wherein alkoxy is C₁-C₆-alkoxy and alkyl is C₁-C₆-alkyl; CO₂(C₁-C₆-alkyl); CO₂H; CH₂OH;
R₄ is selected from hydrogen; C1-C30, preferably C1-C12, more preferably C₁-C₆-alkyl; C2-C30, preferably C2-C12, more preferably C₂-C₆-alkenyl; C2-C30, preferably C2-C12, more preferably C₂-C₆-alkynyl; C4-C30, preferably C₄-C₁₆-cycloalkylalkyl, wherein cycloalkyl preferably is C₃-C₁₀-cycloalkyl and alkyl preferably is C₁-C₆-alkyl; C5-C30, preferably C₅-C₁₆-cycloalkylalkenyl, wherein cycloalkyl preferably is C₃-C₁₀-cycloalkyl and alkenyl preferably is C₂-C₆-alkenyl; C5-C30, preferably C₅-C₁₆-cycloalkylalkynyl, wherein cycloalkyl preferably is C₃-C₁₀-cycloalkyl and alkynyl preferably is C₂-C₆-alkynyl; C2-C30, preferably C2-C12, more preferably C₂-C₆-alkenyl; C2-C12, preferably C₂-C₆-alkynyl; C7-C30, preferably C₇-C₁₆-arylalkyl, wherein aryl preferably is C₆-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; C8-C30, preferably C₈-C₁₆-arylalkynyl, wherein aryl preferably is C₆-C₁₀-aryl and alkynyl preferably is C₂-C₆-alkynyl; C2-C30, preferably C2-C12, more preferably C₂-C₆-alkanoyl; C3-C30, preferably C3-C12, more preferably C₃-C₆-alkenoyl; C3-C30, preferably C3-C12, more preferably C₃-C₆-alkinoyl; C7-C30, preferably C₇-C₁₆-arylalkanoyl, wherein aryl preferably is C₆-C₁₀-aryl and alkanoyl preferably is C₁-C₆-alkanoyl; C9-C30, preferably C₉-C₁₆-arylalkenoyl, wherein aryl preferably is C₆-C₁₀-aryl and alkenoyl preferably is C₃-C₆-alkenoyl; C9-C30, preferably C₉-C₁₆-arylalkinoyl, wherein aryl preferably is C₆-C₁₀-aryl and alkinoyl preferably is C₃-C₆-alkinoyl; iminomethyl, formamidinyl, C1-C30, preferably C1-C12, more preferably C₁-C₆—N-alkyl- and N,N′-dialkylformamidinyl; C2-C30, preferably C2-C12, more preferably C₂-C₆—N-alkenyl- and N,N′-dialkenylformamidinyl; C2-C30, preferably C2-C12, more preferably C₂-C₆—N-alkynyl- and N,N′-dialkynylformamidinyl; C4-C30, preferably C₄-C₁₆—N-cycloalkylalkyl- and N,N′-dicycloalkylalkylformamidinyl, wherein cycloalkyl preferably is C₃-C₁₀-cycloalkyl and alkyl preferably is C₁-C₆-alkyl; C5-C30, preferably C₅-C₁₆—N-cylcoalkylalkenyl- and N,N′-dicycloalkylalkenylformamidinyl, wherein cycloalkyl preferably is C₃-C₁₀-cycloalkyl and alkenyl preferably is C₂-C₆-alkenyl; C5-C30, preferably C₅-C₁₆—N-cycloalkylalkynyl- and N,N″-dicycloalkylalkynylformamidinyl, wherein cycloalkyl preferably is C₃-C₁₀-cycloalkyl and alkynyl preferably is C₂-C₅-alkynyl; C7-C30, preferably C₇-C₁₆—N-arylalkyl- and N,N′-diarylalkylformamidinyl, wherein aryl preferably is C₆-C₁₀-aryl and alkyl preferably is C₁-C₆-alkyl;
R₅ is selected from hydrogen, C1-C30, preferably C1-C12, more preferably C₁-C₆-alkyl; C2-C30, preferably C2-C12, more preferably C₂-C₆-alkenyl; C2-C30, preferably C2-C12, more preferably C₂-C₆-alkynyl; C4-C30, preferably C₄-C₁₆-cycloalkylalkyl, wherein cycloalkyl preferably is C₃-C₁₀-cycloalkyl and alkyl preferably is C₁-C₆-alkyl; C5-C30, preferably C₅-C₁₆-cycloalkylalkenyl, wherein cycloalkyl preferably is C₃-C₁₀-cycloalkyl and alkenyl preferably is C₂-C₆-alkenyl; C5-C30, preferably C₅-C₁₆-cycloalkylalkynyl, wherein cycloalkyl preferably is C₃-C₁₀-cycloalkyl and alkynyl preferably is C₂-C₆-alkynyl; C7-C30, preferably C₇-C₁₆-arylalkyl, wherein aryl preferably is C₅-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; C8-C30, preferably C₈-C₁₆-arylalkynyl, wherein aryl preferably is C₆-C₁₀-aryl and alkynyl preferably is C₂-C₆-alkynyl;
CH(A)CO₂B, wherein A is selected from hydrogen; hydroxyl; C1-C30, preferably C1-C12, more preferably C₁-C₆-alkyl; C2-C30, preferably C2-C12, more preferably C₂-C₆-alkenyl; C2-C30, preferably C2-C12, more preferably C₂-C₆-alkynyl; C4-C30, preferably C₄-C₁₆-cycloalkylalkyl, wherein cycloalkyl preferably is C₃-C₁₀-cycloalkyl and alkyl preferably is C₁-C₆-alkyl; C5-C30, preferably C₅-C₁₆-cycloalkylalkenyl, wherein cycloalkyl preferably is C₃-C₁₀-cycloalkyl and alkenyl preferably is C₂-C₆-alkenyl; C5-C30, preferably C₅-C₁₆-cycloalkylalkynyl, wherein cycloalkyl preferably is C₃-C₁₀-cycloalkyl and alkynyl preferably is C₂-C₆-alkynyl; C7-C30, preferably C₇-C₁₆-arylalkyl, wherein aryl preferably is C₆-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; C8-C30, preferably C₈-C₁₆-arylalkynyl, wherein aryl preferably is C₆-C₁₀-aryl and alkynyl preferably is C₂-C₆-alkynyl; amino; C1-C30, preferably C1-C12, more preferably C₁-C₆-alkylamino; guanidino; C1-C30, preferably C1-C12, more preferably C₁-C₆-alkyl-CO₂B; C₁-C₆-monohydroxyalkyl; C₂-C₆-dihydroxyalkyl; C₃-C₆-trihydroxyalkyl; C₁-C₆-monoaminoalkyl; C₂-C₆-diaminoalkyl; C₃-C₆-triaminoalkyl; C₁-C₆-alkylguanidino; C₁-C₆-alkylcarboxamide; C₁-C₆-alkylhydroxycarbonyl; C₁-C₆-sulfhydrylalkyl; C₂-C₁₂-alkylthioalkyl, wherein alkylthio is preferably C₁-C₆ and alkyl is preferably C₁-C₆; and wherein B is selected from hydrogen; C1-C30, preferably C1-C12, more preferably C₁-C₆-alkyl; C2-C30, preferably C2-C12, more preferably C₂-C₆-alkenyl; C2-C30, preferably C2-C12, more preferably C₂-C₆-alkynyl; C4-C30, preferably C₄-C₁₆-cycloalkylalkyl, wherein cycloalkyl preferably is C₃-C₁₀-cycloalkyl and alkyl preferably is C₁-C₆-alkyl; C5-C30, preferably C₅-C₁₆-cycloalkylalkenyl, wherein cycloalkyl preferably is C₃-C₁₀-cycloalkyl and alkenyl preferably is C₂-C₆-alkenyl; C5-C30, preferably C₅-C₁₆-cycloalkylalkynyl, wherein cycloalkyl preferably is C₃-C₁₀-cycloalkyl and alkynyl preferably is C₂-C₆-alkynyl; C7-C30, preferably C₇-C₁₆-arylalkyl, wherein aryl preferably is C₅-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; C8-C30, preferably C₈-C₁₆-arylalkynyl, wherein aryl preferably is C₆-C₁₀-aryl and alkynyl preferably is C₂-C₆-alkynyl; phenyl; substituted phenyl; (C₁-C₆-alkyl) CONH₂; (C₁-C₆-alkyl)CONH(C₁-C₆-alkyl); (C₁-C₆-alkyl)CONH(C₁-C₆-alkyl-CONH₂); (C₁-C₆-alkyl)CONHC(C₁-C₆-alkylOH)₃; (C₁-C₆-alkyl)CON(C₁-C₆-alkyl) (heterocyclic ring); (C₁-C₆-alkyl)CONH-heterocyclic ring; (C₁-C₆-alkyl)CON(C₁-C₆-alkyl-OH)₂; (C₁-C₆-alkyl)CON(C₁-C₆-alkyl-OH—C₁-C₆-alkyl)₂; (C₁-C₆-alkyl)CON(C₁-C₆-alkyl) (C₁-C₆-alkyl-CONH₂); (C₁-C₆-alkyl)CON(C₁-C₆-alkyl-OH—C₁-C₆-alkyl)₂; (C₁-C₆-alkyl)CON(C₁-C₆-alkyl)₂; (C₁-C₆-alkyl)CON(C₁-C₆-alkyl)₂; (C₁-C₆-alkyl)CONHCO(C₁-C₆-alkyl); (C₁-C₆-alkyl)CON(C₁-C₆-alkyl)N(C₁-C₆-alkyl)₂; (C₁-C₆-alkyl)CON(C₁-C₆-alkyl)CO(C₁-C₆-alkyl); (C₁-C₆-alkyl)CON(C₁-C₆-alkyl)N(C₁-C₆-alkyl)₂; (C₁-C₆-alkyl)COO(C₁-C₆-alkyl); (C₁-C₆-alkyl)N(C₁-C₆-alkyl)₂; (C₁-C₆-alkyl)N(C₁-C₆-alkyl)CO(C₁-C₆-alkyl); (C₁-C₆-alkyl)OCO(C₁-C₆-alkyl); (C₁-C₆-alkyl)OO(C₁-C₆-alkyl); (C₁-C₆-alkyl)COO(C₁-C₆-alkyl); (C₁-C₆-alkyl) CO₂H; (C₁-C₆-alkyl)OCOO(C₁-C₆-alkyl); (C₁-C₆-alkyl)S(C₁-C₆-alkyl); (C₁-C₆-alkyl)SO(C₁-C₆-alkyl); (C₁-C₆-alkyl)SO₂(C₁-C₆-alkyl); phthalidyl, (5-methyl-2-oxo-1,3-dioxol-4-yl)methyl;
CH(A)SO₃B, wherein A and B are defined as above;
CH(A)COB1, wherein A is defined as above, and B1 is NH₂; NH—C1-C12, more preferably NH—C₁-C₆-alkyl; N—(C1-C12)₂, more preferably N—(C₁-C₆)₂-alkyl;
R₆ is selected from CH(A)CO₂B, wherein A is defined as above, and wherein B is selected from C1-C30, preferably C1-C12, more preferably C₁-C₆-alkyl; C2-C30, preferably C2-C12, more preferably C₂-C₆-alkenyl; C2-C30, preferably C2-C12, more preferably C₂-C₆-alkynyl; C4-C30, preferably C₄-C₁₆-cycloalkylalkyl, wherein cycloalkyl preferably is C₃-C₁₀-cycloalkyl and alkyl preferably is C₁-C₆-alkyl; C5-C30, preferably C₅-C₁₆-cycloalkylalkenyl, wherein cycloalkyl preferably is C₃-C₁₀-cycloalkyl and alkenyl preferably is C₂-C₆-alkenyl; C5-C30, preferably C₅-C₁₆-cycloalkylalkynyl, wherein cycloalkyl preferably is C₃-C₁₀-cycloalkyl and alkynyl preferably is C₂-C₆-alkynyl; C7-C30, preferably C₇-C₁₆-arylalkyl, wherein aryl preferably is C₅-C₁₀-aryl and alkyl preferably is C₁-C₆-alkyl; C8-C30, preferably C₈-C₁₆-arylalkenyl, wherein aryl preferably is C₆-C₁₀-aryl and alkenyl preferably is C₂-C₆-alkenyl; C8-C30, preferably C₈-C₁₆-arylalkynyl, wherein aryl preferably is C₆-C₁₀-aryl and alkynyl preferably is C₂-C₆-alkynyl; phenyl; substituted phenyl; (C₁-C₆-alkyl)CONH₂; (C₁-C₆-alkyl)CONH(C₁-C₆-alkyl); (C₁-C₆-alkyl)CONH(C₁-C₆-alkyl-CONH₂); (C₁-C₆-alkyl)CONHC(C₁-C₆-alkylOH)₃; (C₁-C₆-alkyl)CON(C₁-C₆-alkyl) (heterocyclic ring); (C₁-C₆-alkyl) CONH-heterocyclic ring; (C₁-C₆-alkyl)CON(C₁-C₆-alkyl-OH)₂; (C₁-C₆-alkyl)CON(C₁-C₆-alkyl-OH—C₁-C₆-alkyl)₂; (C₁-C₆-alkyl)CON(C₁-C₆-alkyl) (C₁-C₆-alkyl-CONH₂); (C₁-C₆-alkyl)CON(C₁-C₆-alkyl-OH—C₁-C₆-alkyl)₂; (C₁-C₆-alkyl)CON(C₁-C₆-alkyl)₂; (C₁-C₆-alkyl)CON(C₁-C₆-alkyl)₂; (C₁-C₆-alkyl)CONHCO(C₁-C₆-alkyl); (C₁-C₆-alkyl)CON(C₁-C₆-alkyl)N(C₁-C₆-alkyl)₂; (C₁-C₆-alkyl)CON(C₁-C₆-alkyl)CO(C₁-C₆-alkyl); (C₁-C₆-alkyl)CON(C₁-C₆-alkyl)N(C₁-C₆-alkyl)₂; (C₁-C₆-alkyl)N(C₁-C₆-alkyl)₂; (C₁-C₆-alkyl)N(C₁-C₆-alkyl)CO(C₁-C₆-alkyl); (C₁-C₆-alkyl)OCO(C₁-C₆-alkyl); (C₁-C₆-alkyl)OO(C₁-C₆-alkyl); (C₁-C₆-alkyl)COO(C₁-C₆-alkyl); (C₁-C₆-alkyl) CO₂H; (C₁-C₆-alkyl)OCOO(C₁-C₆-alkyl); (C₁-C₆-alkyl)S(C₁-C₆-alkyl); (C₁-C₆-alkyl)SO(C₁-C₆-alkyl); (C₁-C₆-alkyl)SO₂(C₁-C₆-alkyl); phthalidyl, (5-methyl-2-oxo-1,3-dioxol-4-yl)methyl;
CH(A)SO₃B, wherein A and B are defined as above; and
CH(A)COB1, wherein A is defined as above, and B1 is NH₂; NH—C1-C12, more preferably NH—C₁-C₆-alkyl; N—(C1-C12)₂, more preferably N—(C₁-C₆)₂-alkyl.

15. Compound according to claim 14 for the treatment of pain.

16. Compound according to claim 14, for the treatment of gastric diseases (inflammation of the stomach, gastric ulcers), intestinal diseases, particularly chronic inflammation of the small and large intestines (irritable colon syndrome—colon irritabile, colitis ulcerosa, Morbus Crohn), diarrhea, constipation and ileus; rheumatic diseases such as rheumatoid arthritis, osteoarthritis, arthrosis, spondylosis, lumbago, lupus erythematosus and spondylarthropathy; tumors and cancer; obesity and overweight; hepatic disorders, and liver inflammatory disorders.

17. Compound according to claim 14, for the withdrawal of drug addiction, such as to opiates, cocaine, alcohol, for the withdrawal of food, buying, Internet, computer, phone and gambling addiction, for the treatment of psychic diseases, psychosis, schizophrenia, stress-related conditions (such as depression and anxiety), eating disorders and for the reduction of food intake in humans.
